SEQUENCE LISTING (17 sequences)

SEQ ID NO: 1 - DNA, 30 bp, Artificial Sequence
  Description of Artificial Sequence Synthetic primer
cgcggatccatgggtgctgacgacgtcgtc

SEQ ID NO: 2 - DNA, 33 bp, Artificial Sequence
  Description of Artificial Sequence Synthetic primer
cgcggatccttaggcttgagccatatactcata

SEQ ID NO: 3 - DNA, 45 bp, Artificial Sequence
  Description of Artificial Sequence Synthetic primer
gctgagggttcttctagcgttgattatattaataactgggaacag

SEQ ID NO: 4 - DNA, 45 bp, Artificial Sequence
  Description of Artificial Sequence Synthetic primer
ctgttcccagttattaatataatcaacgctagaagaaccctcagc

SEQ ID NO: 5 - DNA, 45 bp, Artificial Sequence
  Description of Artificial Sequence Synthetic primer
gctgagggttcttctagcgtttcctatattaataactgggaacag

SEQ ID NO: 6 - DNA, 45 bp, Artificial Sequence
  Description of Artificial Sequence Synthetic primer
ctgttcccagttattaatataggaaacgctagaagaaccctcagc

SEQ ID NO: 7 - DNA, 45 bp, Artificial Sequence
  Description of Artificial Sequence Synthetic primer
gaagagttcatcaagagattcgatgacggtgcttccagagtcgtc

SEQ ID NO: 8 - DNA, 45 bp, Artificial Sequence
  Description of Artificial Sequence Synthetic primer
gacgactctggaagcaccgtcatcgaatctcttgatgaactcttc

SEQ ID NO: 9 - DNA, 30 bp, Artificial Sequence
  Description of Artificial Sequence Synthetic primer
ccggaattcatggtgaacttcacggtagac

SEQ ID NO: 10 - DNA, 31 bp, Artificial Sequence
  Description of Artificial Sequence Synthetic primer
ccgctcgagctacaatttgtccaggaagttg

SEQ ID NO: 11 - DNA, 42 bp, Artificial Sequence
  Description of Artificial Sequence Synthetic primer
cacgccgacgccatccaccgccgagggggccagatcatcccc

SEQ ID NO: 12 - DNA, 42 bp, Artificial Sequence
  Description of Artificial Sequence Synthetic primer
ggggatgatctggccccctcggcggtggatggcgtcggcgtg

SEQ ID NO: 13 - protein, 522 aa, Homo sapiens
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 14 - protein, 186 aa, Cornyebacterium diphtheria
MGADDVVDSSKSFVMENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQA

SEQ ID NO: 15 - protein, 172 aa, Cornyebacterium diphtheria
MENFASYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQA

SEQ ID NO: 16 - DNA, 519 bp, Cornyebacterium diphtheria
atggagaacttcgcttcctaccacgggaccaagccaggttacgtcgactccatccagaagggtatccagaagccaaagtccggcacccaaggtaactacgacgacgactggaaggggttctactccaccgacaacaagtacgacgctgcgggatactctgtagataatgaaaacccgctctctggaaaagctggaggcgtggtcaaggtcacctacccaggtctgactaaggtcttggctttgaaggtcgacaacgctgagaccatcaagaaggagttgggtttgtccttgactgagccattgatggagcaagtcggtaccgaagagttcatcaagagattcggtgacggtgcttccagagtcgtcttgtccttgccattcgctgagggttcttctagcgttgaatatattaataactgggaacaggctaaggctttgtctgttgaattggagattaacttcgaaaccagaggtaagagaggtcaagatgcgatgtatgagtatatggctcaagcctaa

SEQ ID NO: 17 - protein, 858 aa, Homo sapiens
MVNFTVDQIRAIMDKKANIRNMSVIAHVDHGKSTLTDSLVCKAGIIASARAGETRFTDTRKDEQERCITIKSTAISLFYELSENDLNFIKQSKDGAGFLINLIDSPGHVDFSSEVTAALRVTDGALVVVDCVSGVCVQTETVLRQAIAERIKPVLMMNKMDRALLELQLEPEELYQTFQRIVENVNVIISTYGEGESGPMGNIMIDPVLGTVGFGSGLHGWAFTLKQFAEMYVAKFAAKGEGQLGPAERAKKVEDMMKKLWGDRYFDPANGKFSKSATSPEGKKLPRTFCQLILDPIFKVFDAIMNFKKEETAKLIEKLDIKLDSEDKDKEGKPLLKAVMRRWLPAGDALLQMITIHLPSPVTAQKYRCELLYEGPPDDEAAMGIKSCDPKGPLMMYISKMVPTSDKGRFYAFGRVFSGLVSTGLKVRIMGPNYTPGKKEDLYLKPIQRTILMMGRYVEPIEDVPCGNIVGLVGVDQFLVKTGTITTFEHAHNMRVMKFSVSPVVRVAVEAKNPADLPKLVEGLKRLAKSDPMVQCIIEESGEHIIAGAGELHLEICLKDLEEDHACIPIKKSDPVVSYRETVSEESNVLCLSKSPNKHNRLYMKARPFPDGLAEDIDKGEVSARQELKQRARYLAEKYEWDVAEARKIWCFGPDGTGPNILTDITKGVQYLNEIKDSVVAGFQWATKEGALCEENMRGVRFDVHDVTLHADAIHRGGGQIIPTARRCLYASVLTAQPRLMEPIYLVEIQCPEQVVGGIYGVLNRKRGHVFEESQVAGTPMFVVKAYLPVNESFGFTADLRSNTGGQAFPQCVFDHWQILPGDPFDNSSRPSQVVAETRKRKGLKEGIPALDNFLDKL

(19) United States
(12) Patent Application Publication
(10) Pub. No.: US 2011/0104789 A1
(43) Pub. Date: May 5, 2011

(54) NON-INTEGRATING REV-DEPENDENT LENTIVIRAL VECTOR AND METHODS OF USING THE SAME

(76) Inventors: Yuntao Wu, Manassas, VA (US); Zhirui Wang, Malden, MA (US)

(21) Appl. No.: 12/916,849

(22) Filed: Nov. 1, 2010

Related U.S. Application Data
(60) Provisional application No. 61/256,432, filed on Oct. 30, 2009.

(51) Int. Cl. C12N 7/00; C12N 5/10; C12N 5/00; C07H 21/04; C12N 15/63

(52) U.S. Cl. 435/235.1; 435/366; 435/375; 536/23.1; 435/320.1

(57) ABSTRACT

Non-integrating, Rev-dependent (NIRD) lentiviral vectors and NIRD lentiviral particles carrying a therapeutic gene, such as DT-A or TRAF6 and methods of making the same are disclosed. The intracellular expression of DT-A or TRAF6 results in the selective killing of HIV-positive cells and, thus, these NIRD lentiviral vectors and lentiviral particles can be used in methods to kill HIV-infected cells or treat to HIV-infected subjects. Also disclosed is a human cell line comprising a mutation in the EF2 gene that confers resistance to DT-A.

### RELATED APPLICATIONS

This application claims priority to U.S. Provisional Patent Application No. 61/256,432, filed Oct. 30, 2009, which is hereby incorporated by reference in its entirety.

### GOVERNMENT INTEREST

The present invention was made with government support under Grant Number NS051130 and awarded by the National Institute of Health/NINDS. The U.S. Government has certain rights in the invention.

### BACKGROUND

The human immunodeficiency virus (HIV), remains a global pandemic despite the development of antiretroviral drugs targeting HIV. As of 2007, it was estimated that more than 33 million people were infected with HIV, and HIV associated diseases represent a major world health problem. HIV is a retrovirus that infects certain cells of the immune system, including CD4⁺ T cells and macrophages, destroying or impairing their function. As the infection progresses, the immune system becomes weaker, leaving the infected person more susceptible to opportunistic infections and tumors, such as Kaposi's sarcoma, cervical cancer, lymphoma, and neurological disorders. The most advanced stage of HIV infection is acquired immunodeficiency syndrome (AIDS). It can take 10-15 years for an HIV-infected person to develop AIDS. Certain antiretroviral drugs can delay the process even further.

The development of highly active anti-retroviral therapy (HAART) has allowed for effective control of HIV-1 replication and a reduction in mortality from AIDS⁵⁹. However, the success of HAART is associated with significant setbacks such as toxic side effects, high pill burden, and the development of viral resistance. More importantly, HAART does not completely eliminate HIV from the body, resulting in viral latency and low-level replication in T cells and macrophages permit viral persistence¹⁻³. As a result, patients have to be on drugs for a lifetime: if treatment stops, residual viral reservoirs expand rapidly, allowing disease to progress^{24,60}.

The identification and characterization of these viral reservoirs have highlighted the limitations of HAART, which is often incapable of eliminating the pool of persistently infected cells⁴⁻¹². HIV can be stably maintained in a variety of cells such as brain macrophages¹³, blood monocytes and tissue macrophages^{2,3,10,11,14,15,16}, as well as resting CD4 T cells^{6-8,17}. These reservoirs are either less sensitive to antiviral drugs because of the presence of natural barriers¹⁸, or do not respond to drug treatment because of the absence of viral activity¹². Furthermore, counter to the initial optimism that HAART would lead to immune system recovery^{19,20}, multiple clinical cohort studies have revealed that even though HAART can reduce the viral load to undetectable levels, this is not necessarily followed by full recovery of the immune functions (for a review, see²¹). It appears that the immune system in HIV patients remains impaired and is therefore often unable to mount adequate anti-HIV immune responses, leading to frequent viral rebounds upon HAART discontinuation²²⁻²⁴.

in a previous report, a Rev-dependent lentiviral vector carrying anthrolysin O (AnlO) to target HIV-infected cells was constructed²⁵. It was demonstrated that anlO-mediated cell killing was exclusively dependent on Rev, a unique HIV protein present only in infected cells. Intracellular expression and oligomerization of AnlO resulted in membrane pore formation and cytolysis. In a proof-of-concept study, it was demonstrated that the Rev-dependent AnlO lentivirus specifically diminishes HIV-positive macrophages and T cells. Nevertheless, certain efficacy and safety issues limit the potential for in vivo application of this system. Firstly, AnlO is not very effective since 30 or more molecules are required in order to kill a cell²⁶. Secondly, AnlO kills cells by cytolysis and releases cellular contents into the environment, which may cause inflammation and bystander killing of healthy cells. Thirdly, permanent integration of a suicidal toxin gene, like AnlO, into the human genome threatens to disrupt normal cellular function and cause mutagenesis^{27,28}, especially given that considerable amounts of viral particles may need to be injected into the body.

A novel strategy to specifically target persistently infected cells is urgently needed to improve treatment options for individuals infected with lentiviruses, such as HIV.

### SUMMARY

The present disclosure provides non-integrating, Rev-dependent (NIRD) lentiviral vectors carrying a therapeutic gene. It also provides NIRD lentiviral particles carrying a therapeutic gene, such as DT-A or TRAF6. The intracellular expression of the therapeutic gene results in the selective killing of HIV-positive cells and, thus, these NIRD lentiviral vectors and lentiviral particles can be used in methods to kill HIV-infected cells or treat to HIV-infected subjects. In one embodiment, the therapeutic gene encodes a cytotoxic, cytolytic, or cell apoptosis inducing protein. In another embodiment, the therapeutic gene encodes diphtheria toxin A (DT-A) or TRAF6.

One aspect of the disclosure is directed to a lentiviral particle comprising:

a) a nucleic acid molecule comprising:
i) a promoter, wherein the activity of the promoter is dependent on the presence of a human immunodeficiency virus (HIV) Tat protein;
ii) at least one splice donor site and at least one splice acceptor site;
iii) a nucleotide sequence comprising a therapeutic gene, wherein at least part of the nucleotide sequence is located in an intron between the at least one splice acceptor site and the at least one donor acceptor site; and
iv) a Rev Responsive Element (RRE) from a HIV,
   wherein elements i)-iv) are operably linked;

b) a reverse transcriptase;

c) one or more lentiviral proteins selected from a matrix protein, a capsid protein, a nucleocapsid protein, Vif, Vpr, Vpu, Nef, and Tat; and

d) a mutant integrase, wherein the mutant integrase cannot integrate the nucleic acid molecule into a host cell genome.

In one embodiment, the therapeutic gene encodes a cytotoxic, cytolytic, or cell apoptosis inducing protein. In another embodiment, the therapeutic gene encodes diphtheria toxin A (DT-A) or human TRAF6.

In one embodiment, the lentiviral particle is an HIV particle. In another embodiment, the lentiviral particle is a simian immunodeficiency virus (SIV) particle or a feline immunodeficiency virus (FIV) particle.

In one embodiment, the reverse transcriptase is encoded by an HIV pol gene. In another embodiment, the mutant integrase comprises a mutation at amino acid 116 of the integrase encoded by the HIV pol gene.

Another aspect of the disclosure is directed to a method of producing a lentiviral particle, as described herein, and the lentiviral particles produced according to the method. Specifically, the method of producing a lentiviral particle comprises transfecting into a host cell under conditions permitting the production of the lentiviral particle:

a) a first vector comprising a nucleic acid molecule comprising:
i) a promoter, wherein the activity of the promoter is dependent on the presence of the human immunodeficiency virus (HIV) Tat protein;
ii) at least one splice donor site and at least one splice acceptor site;
iii) a first nucleotide sequence comprising a therapeutic gene, wherein at least part of the first nucleotide sequence is located in an intron between the at least one splice acceptor site and the at least one donor acceptor site; and
iv) a Rev Responsive Element (RRE) from the HIV,
   wherein elements i)-iv) are operably linked;

b) a second vector comprising a second nucleotide sequence comprising a lentiviral gag gene and a lentiviral pol gene, wherein the lentiviral pol gene encodes a mutant integrase and wherein the mutant integrase cannot integrate the nucleic acid molecule into the host cell genome; and

c) a third vector comprising a third nucleotide sequence encoding a viral envelope protein.

In one embodiment, the therapeutic gene encodes a cytotoxic, cytolytic, or cell apoptosis inducing protein. In another embodiment, the therapeutic gene encodes diphtheria toxin A (DT-A) or human TRAF6.

In one embodiment of the method, the second nucleotide sequence of the second vector further comprises one or more lentiviral genes selected from vif, vpr, vpu, vpx, tat, nef, and tat.

In another embodiment, the method of producing a lentiviral particle comprises transfecting into a packaging cell line under conditions permitting the production of the lentiviral particle a first vector comprising a nucleic acid molecule comprising:
i) a promoter, wherein the activity of the promoter is dependent on the presence of the human immunodeficiency virus (HIV) Tat protein;
ii) at least one splice donor site and at least one splice acceptor site;
iii) a first nucleotide sequence comprising a therapeutic gene, wherein at least part of the first nucleotide sequence is located in an intron between the at least one splice acceptor site and the at least one donor acceptor site; and
iv) a Rev Responsive Element (RRE) from the HIV,
   wherein elements i)-iv) are operably linked and wherein the genome of the packaging cell line comprises a viral envelope gene, a lentiviral gag gene, and a lentiviral pol gene, wherein the lentiviral pol gene encodes a mutant integrase and wherein the mutant integrase cannot integrate the nucleic acid molecule into the genome of the packaging cell line.

In one embodiment, the therapeutic gene encodes a cytotoxic, cytolytic, or cell apoptosis inducing protein. In another embodiment, the therapeutic gene encodes diphtheria toxin A (DT-A) or human TRAF6.

In one embodiment, the methods above further comprise recovering the viral particles produced by the host cell or the packaging cell line.

In another embodiment of the methods above, the therapeutic gene encodes DT-A and the host cell or the packaging cell line comprises a mutant human EF2 gene that confers DT-A resistance to the host cell or the packaging cell line.

In another embodiment of the methods above, the therapeutic gene encodes a mutant DT-A, wherein the mutant DT-A is less toxic than the wild type DT-A.

In another embodiment of the methods above, the viral particle is an HIV particle.

In another embodiment of the methods above, the viral envelope protein is a vesicular stomatitis virus G protein. In yet another embodiment of the methods above, the lentiviral gag gene is an HIV gag gene and the lentiviral pol gene is an HIV pol gene.

Another aspect is directed to an isolated human host cell comprising a mutant human EF2 gene, wherein the mutant human EF2 gene comprises a mutation that confers resistance to diphtheria toxin A. In one embodiment, the mutant human EF2 gene comprises the amino acid sequence of SEQ ID NO. 17 except for a substitution at amino acid 717. In another embodiment, the substitution at amino acid 717 is a glycine to an arginine. In yet another embodiment, the host cell further comprises a nucleic acid molecule comprising:

a) a promoter, wherein the activity of the promoter is dependent on the presence of the human immunodeficiency virus (HIV) Tat protein;

b) at least one splice donor site and at least one splice acceptor site;

c) a nucleotide sequence encoding DT-A, wherein at least part of the nucleotide sequence is located in an intron between the at least one splice acceptor site and the at least one donor acceptor site; and

d) a Rev Responsive Element (RRE) from the HIV,

wherein elements a)-d) are operably linked.

In yet another aspect, the lentiviral particles described herein are used in a method of killing a cell infected with a lentivirus, the method comprising contacting the cell with the lentiviral particle as described herein. In one embodiment, the lentivirus is HIV. In another aspect, the lentiviral particles described herein are used in a method of treating a lentiviral infection in a subject, the method comprising administering to said subject a therapeutically effective amount of a lentiviral particle, as described herein. In one embodiment, the lentiviral infection is an HIV infection.

Another aspect is directed to an isolated nucleic acid molecule comprising:

a) a promoter, wherein the activity of the promoter is dependent on the presence of the human immunodeficiency virus (HIV) Tat protein;

b) at least one splice donor site and at least one splice acceptor site;

c) a nucleotide sequence encoding human TRAF6 or DT-A, wherein at least part of the nucleotide sequence is located in an intron between the at least one splice acceptor site and the at least one donor acceptor site; and

d) a Rev Responsive Element (RRE) from the HIV,

wherein elements a)-d) are operably linked.

In one embodiment, the DT-A is a mutant wherein the mutant DT-A is less toxic than the wild type DT-A. The nucleic acid molecule may optionally be incorporated into a vector.

In one embodiment, the promoter comprises an HIV 5′ long terminal repeat (LTR) or a portion thereof. In another embodiment, the nucleic acid molecule further comprises an HIV 3′ LTR or a portion thereof. In another embodiment, the nucleic acid molecule further comprises a packaging signal. In certain embodiments, the at least one splice donor site is the HIV D1 splice donor site and the at least one splice acceptor site is the HIV A7 splice acceptor site. In other embodiments, the nucleic acid molecule further comprises at least a second splice donor site, such as the HIV D4 splice donor site, and at least a second splice acceptor site, such as the HIV A5 splice acceptor site.

Any embodiment of the nucleic acid molecule described in this application may also be used in the viral particles, host cells, and methods described herein.

The disclosure also provides a mutant DT-A polypeptide, having an N-terminal truncation, wherein the mutant DT-A polypeptide has the amino acid sequence of SEQ ID NO. 15, as well as a nucleic acid molecule encoding the mutant DT-A polypeptide, including, for example, a nucleic acid molecule having the sequence of SEQ ID NO. 16.

### BRIEF DESCRIPTION OF THE DRAWINGS

The accompanying drawings, which are incorporated in and constitute a part of this specification, illustrate certain embodiments, and together with the written description, serve to explain certain principles of the antibodies and methods disclosed herein.

FIG. 1 shows the specificity of the Rev-dependent lentiviral vectors in mediating HIV-dependent gene expression. (A) Schematic representation of the HIV-1 genome, a Rev-dependent lentiviral construct (pNL-GFP-RRE-SA), and the HIV-1 helper construct, pCMVΔR8.2, in which both the viral package signal (Δψ) and the envelope gene (Env) were deleted. Shown are the HIV-1 5′ LTR, packaging signal (ψ), splice donors (D1, D4) and acceptors (A5, A7), internal ribosome entry site (IRES), Rev Responsive Element (RRE), and 3′ LTR. (B) Rev-dependent GFP expression in cotransfection. HEK293T cells (1 million) were cotransfected with a varied amount of pNL-GFP-RRE-SA (from 0.1 to 3 μg) plus 1 μg of pCMVΔR8.3 or 1 μg of an empty vector, pMSCVneo. GFP expression was measured at 48 hours post cotransfection by flow cytometry. Propidium iodide (P.I.) was added to identify viable GFP-expressing cells. In all cases, cells were cotransfected with an equal amount of DNA (4 ug total) using pMSCVneo to make up the difference. (C) Specificity of the Rev-dependent lentiviral vector in HIV-1-positive T cells. CEM-SS cells were not infected (Cell) or infected with NL4-3.HSA.R+E−(VSV-G) (NL4-3.HSA, 1 μg p24 per million cells), a VSV-G pseudotyped HIV-1 strain with the murine heat-stable antigen CD24 (HSA) gene inserted into the nef region that allows HIV-1-positive cells to be monitored by surface staining of HSA. At 24 hours, cells were superinfected with lentivirus vNL-GFP-RRE-SA (1×, m.o.i. 0.2). For comparison, cells were also singly infected with either vNL-GFP-RRE-SA (No HIV infection) or NL4-3.HSA.R+E−(VSV-G) (NL4-3.HSA). At 72 hours, cells were harvested, stained with a PE-labeled rat monoclonal antibody against mouse CD24 (HSA), and then analyzed on a flow cytometer for both HSA and GFP expression. Isotype staining is not shown.

FIG. 2 shows Rev-dependent killing of HIV-positive cells by DT-A, TRAF6, and AnlO. (A) Schematic representation of the Rev-dependent vectors carrying DT-A, TRAF6, and AnlO, and the helper construct, pCMVΔR8.2, that were used to cotransfect HEK293T or HeLa cells. (B) HeLa or HEK293T cells (1 million) were cotransfected with pCMVΔR8.2 (1 μg) plus pNL-DT-GFP-RRE-SA, pNL-TRAF6-GFP-RRE-SA, pNL-AlnO-GFP-RRE-SA, or pNL-GFP-RRE-SA (3 μg). As controls, these Rev-dependent vectors were identically cotransfected with an empty vector, pMSCVneo (1 μg). Cells were also cotransfected with pCMVΔR8.2 without the Rev-dependent vectors (1 μg pCMVΔR8.2 plus 3 μg pMSCVneo). GFP expression was measured at 48 hours post cotransfection by flow cytometry. Propidium iodide (P.I.) was added to identify viable GFP-expressing cells.

FIG. 3 shows Rev-dependent killing of HIV-positive cells by DT-A mutants. (A) HEK293T cells (1 million) were cotransfected with pCMVΔR8.2 (1 μg) and one of the Rev-dependent constructs carrying the DT-A mutants, pNL-DT(E148S)-GFP-RRE-SA, pNL-DT(E148D)-GFP-RRE-(SA), pNL-DT(176)-GFP-RRE-SA, or pNL-DTΔN-GFP-RRE-(SA) (3 μg). Cell killing was monitored by GFP expression at 48 hours post infection using flow cytometry. (B) The same cotransfection experiments were repeated in a DT-A resistant cell line, 5H7.

FIG. 4 shows additional testing of the DT-A-resistant HEK293T cells. (A) The human EF-2 mutant (G717R) was introduced into HEK293T cells by retroviral vector transduction. Cells were screened for the EF-2 mutation. Originally, 100 clones were selected, and 5 of them turned GFP-positive when cotransfected with pCMVΔR8.2 (1 μg for 1 million cells) plus the DT-A containing Rev-dependent vector, pNL-DT-GFP-RRE-SA (3 μg for 1 million cells). While the parental HEK293T cells generate 0% GFP-positive cells after the cotransfection, the DT-A resistant clones generate GFP-positive cells at different percentages: 46% in 5H7, 28% in CB2, 24% in AB1, 14% in 4H10, and 9% in 5E12, respectively. (B) To further measure the degree of DT-A resistance, one of the clones, 5H7, was cotransfected with pCMVΔR8.2 plus pNL-DT-GFP-RRE-SA. As a control, the cells were also identically cotransfected with pCMVΔR8.2 plus pNL-DT(R)-GFP-RRE-SA in which the DT-A gene was placed in a reverse orientation to prevent protein expression. The parental HEK293T cells were also identically cotransfected with these constructs. As an additional control, cells were not infected (Cell) with the constructs. (C) Western blot analysis of both 5H7 and HEK393T cells cotransfected with either pCMVΔR8.2 plus pNL-DT-GFP-RRE-SA (lanes 2 and 4, DT) or pCMVΔR8.2 plus pNL-DT(R)-GFP-RRE-SA (lanes 1 and 3, DT(R)). Untransfected cells (lanes 5 to 7) and a purified, recombinant DT-A protein (CRM9) (lane 8)⁷⁴ were used as controls. A monoclonal antibody against DT was used for Western blot, and this antibody was also reactivated with a cellular protein (10-15 KD) that was used as the loading control.

FIG. 5 shows the development of the NIRD vector carrying DT-A and TRAF6. (A) Schematic representation of the Rev-dependent vector carrying luciferase, the non-integrating helper construct, pCMVΔR8.2(D116N), and pHCMV-G expressing VSV-G. (B) To demonstrate HIV-dependent expression of reporter genes from the NIRD vector, viral particles vNL-Luc-RRE-SA(D116N) and vNL-Luc-RRE-SA were generated by cotransfection of HEK293T cells with pCMVΔR8.2(D116N) or pCMVΔR8.2 plus pNL-Luc-RRE-SA plus pHCMV-G, and then used to superinfect an HIV-1-positive T cell line, J1.1 or the uninfected, parental Jurkat T cells (0.2 million cells). Luciferase was measured at 48 hours in J1.1 and Jurkat cells following infection. Both J1.1 and Jurkat were stimulated with 50 ng/ml PMA before infection. (C) Rev-dependent killing of HIV-positive cells by NIRD vector carrying DT-A and TRAF6. HeLa cells (1 million) were cotransfected with pCMVΔR8.2(D116N) (1 μg) plus pNL-DT-GFP-RRE-SA or pNL-TRAF6-GFP-RRE-SA or pNL-GFP-RRE-SA (3 μg). GFP expression was measured at 48 hours post cotransfection by flow cytometry. Propidium iodide (P.I.) was added to identify viable GFP-expressing cells. (D) Specific targeting of HIV-1-infected lymphocytes by TRAF6 NIRD vector. Human PBMC (1 million cells) were infected with a replication-competent virus NL4-3.HSA.R+ (10⁴ TCID_{50/Rev-CEM}). Aliquots of the infected cells were then superinfected at days 1, 4 and 7 post HIV infection with vNL-TRAF6-GFP-RRE-SA(D116N) (5 μg p24). HIV-1-positive cells were measured by surface staining of mouse HSA followed by flow cytometry at day 9 post HIV-1 infection. (E) To measure non-specific killing of cells by TRAF6 NIRD vector, HIV-uninfected cells were infected with only vNL-TRAF6-GFP-RRE-SA(D116N) as described in (D) (panel d, f, h). Following infection at day 0, 3 and 6, cells were analyzed one day later by propidium iodide (P.I.) staining and flow cytometry (panel d, f, h, respectively). As controls, cells were also mock infected with medium (panel a, c, e, and g), or treated with puromycin to induce non-specific killing (panel b).

FIG. 6 shows that background luciferase readings in HIV-negative Jurkat cells was derived from residual luciferase in the viral preparation. (A) To determine the background luciferase present in the HIV-negative Jurkat cells during infection with vNL-Luc-RRE-SA(D116N), cells (0.5 million cells) were pre-treated with azidothymidine (AZT) (50 μM) overnight, and then uninfected (lane 1) or infected with vNL-Luc-RRE-SA(D116N) for 2 hours (lane 2). Cells were washed and immediately lysed for Western blot analysis using a goat polyclonal anti-luciferase antibody. The blot was also probed with a goat polyclonal antibody to GAPDH for loading controls. (B) The background luciferase activity present in the vNL-Luc-RRE-SA(D116N) viral preparation was reduced by purifying the virion through anion exchange (Sartobind® Q75) and size-exclusion (Vivaspin® 20 and 500) columns. The relative luciferase activities (RLU) present in virion before and after purification were measured (normalized by virion p24).

### DETAILED DESCRIPTION

Reference will now be made in detail to various exemplary embodiments, examples of which are illustrated in the accompanying drawings. It is to be understood that the following detailed description is provided to give the reader a fuller understanding of certain embodiments, features, and details of aspects of the invention, and should not be interpreted as a limitation of the scope of the invention.

### 1. Definitions

In order that the present invention may be more readily understood, certain terms are first defined. Additional definitions are set forth throughout the detailed description.

The term “therapeutically effective amount” refers to a dosage or amount that is sufficient to kill cells infected with a lentivirus, such as HIV.

As used herein, “HIV” and “human immunodeficiency virus” refer to human immunodeficiency virus 1 and 2 (HIV-1 and HIV-2).

As used herein, the term “operably linked” means that the components described are in a relationship permitting them to function in their intended manner.

The terms “treatment” or “treating” and the like refer to any treatment of any disease or condition in a mammal, e.g. particularly a human or a mouse, and includes inhibiting a disease, condition, or symptom of a disease or condition, e.g., arresting its development and/or delaying its onset or manifestation in the patient or relieving a disease, condition, or symptom of a disease or condition, e.g., causing regression of the condition or disease and/or its symptoms.

The terms “subject,” “host,” “patient,” and “individual” are used interchangeably herein to refer to any mammalian subject for whom diagnosis or therapy is desired, particularly humans.

The term “pharmaceutically acceptable carrier” or “pharmaceutically acceptable excipient” means solvents, dispersion media, coatings, antibacterial agents and antifungal agents, isotonic agents, and absorption delaying agents, and the like, that are compatible with pharmaceutical administration. The use of such media and agents for pharmaceutically active substances is well known in the art.

The term “isolated,” when used in the context of a biological molecule refers to a biological molecule that is substantially free of its natural environment. For instance, an isolated nucleic acid or protein is substantially free of cellular material from the cell or tissue source from which it was derived. The term also refers to preparations where the isolated biological molecule is sufficiently pure for pharmaceutical compositions; or at least 70-80% (w/w) pure; or at least 80-90% (w/w) pure; or at least 90-95% pure; or at least 95%, 96%, 97%, 98%, 99%, or 100% (w/w) pure.

As used in this specification and the appended claims, the singular form “a”, “an” and “the” include plural referents unless the context dictates otherwise. Thus, for example, reference to “a virus” includes a plurality of viruses unless the context dictates otherwise.

This disclosure provides non-integrating, Rev-dependent (NIRD) lentiviral vectors and NIRD lentiviral particles carrying a therapeutic gene to target HIV-infected cells. In one embodiment, the therapeutic gene encodes a cytotoxic, cytolytic, or cell apoptosis inducing protein. In another embodiment, the therapeutic gene encodes diphtheria toxin A (DT-A) or TRAF6. The intracellular expression of therapeutic genes like DT-A or TRAF6 results in the selective killing of HIV-positive cells and, thus, these non-integrating Rev-dependent lentiviral vectors and lentiviral particles can be used in methods to kill HIV-infected cells or to treat HIV-infected subjects.

As noted above, we have previously produced an integrating, Rev-dependent lentiviral vector carrying anthrolysin (AnlO) to target HIV-infected cells. To enhance the efficacy and safety of this vector system, we developed numerous modifications to the vector system.

First, we selected diphtheria toxin A chain (DT-A) as the primary suicide gene to induce cell death. DT is a potent inhibitor of protein synthesis and catalyzes ADP ribosylation of human elongation factor 2 (EF-2), which triggers cell death by apoptosis without the leakage of cellular contents^{29,30}. It has been estimated that a single molecule of DT is sufficient to kill a cell³¹, and the DT-A chain contains the catalytic domain of this enzymatic action. Another major advantage of using DT-A is that a wealth of information is available on this toxin³²⁻³⁴. In particular, multiple human clinical trials have been conducted using DT-fusion proteins for cancer therapy, providing important information about its safety in patients³⁵⁻³⁷.

However, the potency of DT-A presents unique challenges to using this toxin in a Rev-dependent lentiviral vector system. Given the ultimate potency of DT-A, tightly regulated expression, namely the expression of DT-A only in the presence of HIV Rev, is an important regulatory strategy. However, low-level background expression of DT-A resulting from leakage in non-target cells must also be addressed. To counter this low level background expression, we used attenuated DT-A mutants³⁸. In addition, given the potency of DT-A, unwanted killing of producer cells during viral production precludes the assembly of viral particles in vitro. To resolve this major technical hurdle, we developed a DT-resistant human cell line through site-directed mutagenesis of the human EF-2 gene²⁹. Before developing this DT-resistant human cell line, it was not possible to produce viral particles from a Rev-dependent lentiviral expression vector carrying the DT-A gene because expression of DT-A would kill the producer cell before the viral particles could be assembled. The successful development of the DT-resistant cells allowed us to produce the first high-titer DT-A NIRD viral particles to target persistently infected HIV cells.

As a complement to the use of extraneous toxins, we also chose to test an endogenous human protein, TRAF6 (tumor necrosis factor receptor-associated factor 6). Overexpression of TRAF6 induces apoptosis by activation of Caspase 8³⁹. As a self-protein, human TRAF6 has a unique advantage for in vivo application. While high-level expression of TRAF6 can trigger apoptosis, low-level expression would be tolerated by cells and the immune system, minimizing possible side effects from leakage and non-specific expression in non-target cells.

Finally, we also modified the original vector system by developing an integration defective vector. This non-integrating Rev-dependent (NIRD) lentiviral vector was created using a mutant viral integrase present in a non-integrating HIV-1 mutant, D116N⁴⁰. Previously, we demonstrated that low-level transcription occurs from non-integrating HIV DNA both in human T cells and in macrophages, two of the primary HIV targets⁴¹⁻⁴³. We also demonstrated recently that the templates for non-integrating transcription are a large population of viral DNA⁴⁴. Several recent studies have used non-integrating lentiviral vectors for the safer delivery of therapeutic genes for gene therapy, demonstrating the efficacy of the system to express therapeutic genes⁴⁵⁻⁴⁷.

The NIRD vector offers a safety advantage by reducing the possible risk of integration-mediated mutagenesis. Insertional transformation by retroviral vectors has been known to result in malignancy^{27,28} which raises concerns for the safe application of lentiviral vectors for clinical gene therapy, especially given that large quantities of viral particles may need to be injected. The use of integrase mutants, although unable to completely eliminate integration⁶¹, does provide a significant reduction (10³- to 10⁴-fold) in viral integration⁶².

### 2. Lentiviruses

The human immunodeficiency viruses (HIV) are members of the Retroviridae family and, more particularly, are classified within the Lentivirinae subfamily. Other members of the lentivirus family include simian immunodeficiency virus (SIV), and feline immunodeficiency virus (FIV). Like nearly all other viruses, the replication cycles of members of the lentivirus family, commonly known as the retroviruses, include attachment to specific cell receptors, entry into cells, synthesis of proteins and nucleic acids, assembly of progeny virus particles (virions), and release of progeny viruses from the cells. A unique aspect of retrovirus replication is the conversion of the single-stranded RNA genome into a double-stranded DNA molecule (provirus) that must integrate into the genome of the host cell prior to the synthesis of viral proteins and nucleic acids.

Lentivirus virions are enveloped and contain two copies of the genome. The lentiviral genome and the proviral DNA have the three genes found in retroviruses: gag, pol and env. These three genes are flanked by two long terminal repeat (LTR) sequences. The gag gene encodes the internal structural (matrix, capsid and nucleocapsid) proteins; the pol gene encodes the RNA-directed DNA polymerase that converts genomic RNA into DNA (reverse transcriptase), a protease and an integrase; and the env gene encodes viral envelope glycoproteins. The 5′ and 3′ LTR's serve to promote transcription and polyadenylation of the virion RNAs. The LTR contains other cis-acting sequences involved in viral replication. Lentiviruses have additional genes including vif, vpr, tat, rev, vpu, nef and vpx (in HIV-1, HIV-2 and/or SIV). The expression of this unusually high number of gene products is accomplished by use of multiple reading frames and multiple splicing sites.

Transcription from the provirus is regulated by the activity of the HIV promoter, the long terminal repeat (LTR) found at the 5′ end of the DNA, which contains binding sites for numerous cellular transcription factors. In the absence of premature termination, expression from the provirus results in the generation of a single “full length” RNA species. This non-spliced transcript serves as messenger for several HIV structural proteins (gag-pol genes), as well as the RNA genome that is incorporated into newly synthesized HIV particles. There are events in normal HIV infection, however, that precede the accumulation of new genomic RNA. Common for host and retroviral gene expression, co-transcriptional association of the forming message with an assortment of proteins, including splicing enzymes, results in the removal of introns and efficient delivery of the mature message to the cytosol. The full-length HIV transcript also contains a variety of splicing donors and acceptor sites. This feature of HIV permits the encoding of various proteins in overlapping genes (within the same segment of DNA), and permits a temporal separation of gene expression. Through varied use and non-use of splicing sites, the single RNA generated from the integrated DNA can yield nearly forty different transcripts that encode a total of nine different proteins⁷⁵. In the infected cell, the earliest RNA generated becomes fully spliced by the cellular splicing machinery.

Fully spliced HIV transcripts encode three proteins: negative factor Nef, trans-activator of transcription Tat, and the regulator of viral gene expression Rev. These three gene products are regulatory proteins that affect cellular and viral functions that lead to efficient viral replication, but more specifically, all three can alter the viral transcription output. Tat and Rev associate with regions of newly transcribing HIV RNA. Tat associates co-transcriptionally (along with numerous cellular protein factors, including an RNA polymerase II-modifying kinase) with a 5′ stem-loop structure TAR⁷⁶. Tat and the associated proteins function by promoting completion of initiated transcriptional activity (processivity or anti-termination). Rev protein is responsible for the conversion from early HIV gene expression to late gene expression in the newly infected cells. Rev mediates the cytosolic delivery of singly and non-spliced message, and thus its expression coordinates the conversion of predominately Nef, Tat, and Rev (products of multiply spliced transcript) to expression of singly and unspliced HIV transcripts, such as those for the structural proteins of the virion⁷⁷. This occurs through a physical interaction of Rev with unspliced or singly spliced transcripts and with cellular components that are responsible for message export from the nucleus. The RNA region for Rev association, the Rev-responsive element (RRE), is located in the 3′ half of the HIV RNA within the env gene. Multiple copies of Rev assemble on the RRE and a different region of Rev associates with the CRM1 nuclear export protein. This association mediates transport of the transcripts to the cytosol.

### 3. Rev-Dependent Lentiviral Expression Vector

The present disclosure provides nucleic acid molecules comprising expressible sequences, such as DT-A and TRAF6, whose expression is dependent on the presence of viral Rev proteins. Rev-dependent lentiviral expression vectors have been previously described⁴⁹. Generally, the Rev-dependent lentiviral expression vector comprises four segments of the lentiviral genome (e.g., HIV), although no lentiviral gene is expressed from the construct.

One embodiment of the vector comprises the following four segments from the HIV genome. The 5′ end of the vector comprises the 5′ LTR, a first splice donor site (e.g., splice donor site 1; “D1”), and a portion of the gag open reading frame that includes the packaging signal. The second segment is from the tat1/rev1 exon that includes a first splice acceptor site (e.g., splice acceptor site 5; “A5”), and a second splice donor site (e.g., splice donor site 4; “D4”). The third segment is from the env gene and comprises the Rev Response Element (RRE), and a second splice acceptor site (e.g., splice acceptor site 7; “A7”), which is preferably located within the RRE. The RRE renders gene expression dependent on Rev, a viral early protein interacting specifically with RRE to mediate mRNA nuclear export and translation. The fourth segment includes the 3′ LTR and a small portion of the nef reading frame 5′ to the LTR.

The vector also contains sites into which the open reading frame of one or more nucleotide sequences of interest can be inserted. The full-length transcript generated from the vector possesses sufficient splice sites that mediate the removal of the open reading frame(s). Thus, unless Rev is present, the open reading frame of the one or more nucleotide sequences of interest, which is contained within an intron bordered by a splice donor site and a splice acceptor site, is rapidly spliced out by the cellular splicing machinery. On the other hand, in the presence of HIV infected cells, where the Rev protein is present, the singly spliced or non-spliced transcripts are delivered to the cytosol, and the one or more sequences of interest are expressed.

### 4. Production of Non-Integrating Lentiviral Particles

The NIRD lentiviral expression vectors described herein can be used to produce infectious, non-integrating lentiviral particles containing a nucleic acid sequence of interest, such as a nucleotide sequence encoding human TRAF6 or DT-A. One way to produce such infectious, non-integrating lentiviral particles is to use one or more “helper expression vectors” that complement for the inability of the Rev-dependent lentiviral expression vector to form lentiviral particles. Such helper-expression vectors are common and are easily constructed by those of ordinary skill in the art.⁴⁹

For example, it is possible to prepare such infectious, non-integrating lentiviral particles by introducing into a host cell three different nucleotide sequences, i.e., a first nucleotide sequence encoding a viral envelope protein, a second nucleotide sequence comprising a lentiviral gag gene and a lentiviral pol gene, and a third nucleotide sequence comprising the defective lentiviral genome comprising the nucleotide sequence of interest (Rev-dependent lentiviral expression vector). Typically, the three different nucleotide sequences are incorporated into vectors that are introduced into the host cell, for example by transfection.

Alternatively, the sequences encoding Gag, Pol and Env proteins can be introduced into a cell and stably integrated into the cell genome to produce a stable cell line called a packaging cell line. The packaging cell line produces the proteins required for packaging retroviral RNA but it cannot bring about encapsidation due to the lack of a packaging signal (psi). However, when a defective lentiviral genome (Rev-dependent lentiviral expression vector) having a packaging signal (psi) is introduced into the packaging cell line, the helper proteins can package the psi-positive lentiviral vector RNA to produce the recombinant lentiviral particles carrying the nucleotide sequence of interest.

Making the lentiviral particle a non-integrating viral particle requires a pol gene encoding a mutant integrase that cannot integrate the viral nucleic acid into the host cell genome. In one embodiment, the mutant pol gene is an HIV pol gene. In another embodiment, the mutation occurs at amino acid 116 within the D(35)E functional motif of the lentiviral integrase. A single point mutation changing amino acid 116 from Asp to Asn (D116N) has been shown to completely abolish HIV integrase activity without affecting other viral functions such as reverse transcription and nuclear targeting.⁴⁰

For the env gene construct, it is common to pseudotype a lentiviral vector with the env gene from another virus. For example, HIV can be pseudotyped by a variety of retroviral envelope proteins, such as those of murine leukemia viruses (MLVs), human T-cell leukemia virus type 1, Jaagsiekte sheep retrovirus, and avian leukosis-sarcoma virus subgroup A, as well as by some nonretroviral envelopes, including vesicular stomatitis virus G protein (VSV-G), lymphocytic choriomeningitis virus glycoprotein, Mokola virus and rabies virus G proteins, and Ebola virus (Zaire) glycoprotein. Thus, in one embodiment, the env gene is from a lentivirus, such as HIV, SIV, or FIV. In another embodiment, the env gene is from a virus other than a lentivirus, including but not limited to a VSV, a MLV, a human T-cell leukemia virus, a Jaagsiekte sheep retrovirus, an avian leukosis-sarcoma virus, a lymphocytic choriomeningitis virus, a Mokola virus, a rabies virus, or an Ebola virus.

### 5. TRAF6

In this study, we selected human TRAF6 as a suicide gene for testing in the NIRD vector. The human TRAFs are intracellular proteins associated with the tumor necrosis factor receptor (TNF-R)⁶⁶. There are six mammalian TRAF family members (TRAF1-6) that are involved in signal transduction by TNF-R family members as well as some members of the Toll-like receptor (TLR) family and IL-1R. Unlike other TRAFs, which largely mediate signaling from the TNF-R superfamily, TRAF6 also participates in the signaling pathway from the IL-1R/TLR superfamily^{67,68}. TRAF6 also directly induces apoptosis, which results from the capacity of human TRAF6 to interact with and activate caspase 8. Both the C-terminal TRAF domain of human TRAF6, which directly interacts with the death effector domain of pro-caspase 8, and the N-terminal RING domain, which is required for activation of caspase 8, are necessary for apoptotic induction³⁹.

The nucleic acid sequence of the human TRAF6 gene is known and set forth at ACCESSION NM_{—}145803; VERSION NM_{—}145803.1 GI:22027629. The amino acid sequence of human TRAF6 is set forth below:

(SEQ ID NO: 13)
MSLLNCENSCGSSQSESDCCVAMASSCSAVTKDDSVGGTASTGNLSSSF
MEEIQGYDVEFDPPLESKYECPICLMALREAVQTPCGHRFCKACIIKSI
RDAGHKCPVDNEILLENQLFPDNFAKREILSLMVKCPNEGCLHKMELRH
LEDHQAHCEFALMDCPQCQRPFQKFHINIHILKDCPRRQVSCDNCAASM
AFEDKEIHDQNCPLANVICEYCNTILIREQMPNHYDLDCPTAPIPCTFS
TFGCHEKMQRNHLARHLQENTQSHMRMLAQAVHSLSVIPDSGYISEVRN
FQETIHQLEGRLVRQDHQIRELTAKMETQSMYVSELKRTIRTLEDKVAE
IEAQQCNGIYIWKIGNFGMHLKCQEEEKPVVIHSPGFYTGKPGYKLCMR
LHLQLPTAQRCANYISLFVHTMQGEYDSHLPWPFQGTIRLTILDQSEAP
VRQNHEEIMDAKPELLAFQRPTIPRNPKGFGYVTFMHLEALRQRTFIKD
DTLLVRCEVSTRFDMGSLRREGFQPRSTDAGV

In addition to the wild type human TRAF6, it is possible to use mutant TRAF6 nucleic acids and polypeptides comprising one or more mutations to the wild type sequence, provided the mutant TRAF6 retains its apoptotic activity. Thus, in one embodiment, the human TRAF6 is a mutant TRAF6 comprising at least 75%, 80%, 85%, 90%, 95%, 96%, 97%, 98%, or 99% identity to the amino acid sequence of SEQ ID NO. 13, wherein the mutant TRAF6 has apoptotic activity, or a nucleic acid encoding the same.

### 6. Diphtheria Toxin A

Diphtheria toxin is an exotoxin secreted by Cornyebacterium diphtheria, the bacterium that causes diphtheria. The toxin consists of two polypeptide fragments (A and B), which are held together by a disulfide bond. Fragment A (“DT-A”) is the toxic fragment, preventing host cells from carrying out protein synthesis. DT-A is one of the most extensively studied and well-understood bacterial toxin used for therapeutics. Ever since its discovery in the late 1800s, it has been a central focus in the field of toxicology. DT-A is highly potent, highly specific, and has a well-defined mechanism of inhibition^{69,70}, placing DT-A at the top of the list of therapeutic toxins^{71,72}. DT-A has been used in vaccine and therapeutic clinical trials, providing much-needed information about its safe use⁷³.

In one embodiment, the Rev-dependent lentiviral expression vector comprises a nucleotide sequence encoding the wild type DT-A. Wild type DT-A has the following amino acid sequence:

(SEQ ID NO. 14)
MGADDVVDSSKSFVMENFASYHGTKPGYVDSIQKGIQKPKSGTQGNYDD
DWKGFYSTDNKYDAAGYSVDNENPLSGKAGGVVKVTYPGLTKVLALKVD
NAETIKKELGLSLTEPLMEQVGTEEFIKRFGDGASRVVLSLPFAEGSSS
VEYINNWEQAKALSVELEINFETRGKRGQDAMYEYMAQA

In other embodiments, it is desirable to use an attenuated DT-A. In these embodiments, the Rev-dependent lentiviral expression vector comprises a nucleotide sequence encoding a mutant DT-A, wherein the mutant DT-A is less toxic than the wild type DT-A. Methods for measuring the toxicity of DT-A and determining whether the toxicity of a mutant DT-A is less than the toxicity of the wild type DT-A are known in the art, and include, for example, the methods disclosed in this application.

In one embodiment, the mutant DT-A has a mutation at position 149 of SEQ ID NO. 14, such as DT-A (E148S) or DT-A (E148D). The E148S mutant has the same amino acid sequence as wild type DT-A except for a glutamic acid to serine substitution at position 149 of SEQ ID NO. 14, while the E148D mutant has the same amino acid sequence as wild type DT-A except for a glutamic acid to aspartic acid substitution at position 149 of SEQ ID NO. 14. In another embodiment, the mutant DT-A is called DT-A (176) and has the same amino acid sequence as wild type DT-A except for a glycine to aspartic acid substitution at position 129 of SEQ ID NO. 14.

In yet another embodiment, the mutant DT-A is an attenuated DT-A toxin with an N-terminal truncation of the wild type DT-A. In one embodiment, the first 14 amino acids of the wild type DT-A have been removed (DT-AΔN) and the mutant DT-A has the amino acid sequence:

(SEQ ID NO. 15)
MENFASYHGTKPGYVDSIQKGIQKPKSGTQGNYDDDWKGFYSTDNKYDA
AGYSVDNENPLSGKAGGVVKVTYPGLTKVLALKVDNAETIKKELGLSLT
EPLMEQVGTEEFIKRFGDGASRVVLSLPFAEGSSSVEYINNWEQAKALS
VELEINFETRGKRGQDAMYEYMAQA

The present disclosure further provides isolated nucleic acids encoding the mutant DT-A, including the DT-AΔN mutant. The nucleic acids may comprise DNA or RNA and may be wholly or partially synthetic or recombinant. In one embodiment, the nucleic acid encodes a DT-AΔN mutant having an amino acid sequence of SEQ ID NO. 15. In another embodiment, the nucleic acid molecule encoding the DT-AΔN has the following nucleic acid sequence:

(SEQ ID NO. 16)
ATGGAGAACTTCGCTTCCTACCACGGGACCAAGCCAGGTTACGTCGACT
CCATCCAGAAGGGTATCCAGAAGCCAAAGTCCGGCACCCAAGGTAACTA
CGACGACGACTGGAAGGGGTTCTACTCCACCGACAACAAGTACGACGCT
GCGGGATACTCTGTAGATAATGAAAACCCGCTCTCTGGAAAAGCTGGAG
GCGTGGTCAAGGTCACCTACCCAGGTCTGACTAAGGTCTTGGCTTTGAA
GGTCGACAACGCTGAGACCATCAAGAAGGAGTTGGGTTTGTCCTTGACT
GAGCCATTGATGGAGCAAGTCGGTACCGAAGAGTTCATCAAGAGATTCG
GTGACGGTGCTTCCAGAGTCGTCTTGTCCTTGCCATTCGCTGAGGGTTC
TTCTAGCGTTGAATATATTAATAACTGGGAACAGGCTAAGGCTTTGTCT
GTTGAATTGGAGATTAACTTCGAAACCAGAGGTAAGAGAGGTCAAGATG
CGATGTATGAGTATATGGCTCAAGCCTAA

The present disclosure also provides constructs in the form of plasmids, vectors, phagemids, transcription or expression cassettes which comprise at least one nucleic acid encoding a DT-A mutant, such as DT-AΔN (SEQ ID NO. 15). The disclosure further provides a host cell which comprises one or more constructs as above.

Also provided are methods of making the polypeptides encoded by these nucleic acids. The method comprises expressing the encoded polypeptide from the encoding nucleic acid. Expression may be achieved by culturing under appropriate conditions recombinant host cells containing the nucleic acid. Following production by expression a mutant DT-A, such as DT-AΔN, may be isolated and/or purified using any suitable technique, then used as appropriate.

### 7. Human Cell Line Resistant to DT-A

As noted above, a major technical hurdle in producing a Rev-dependent lentiviral expression vector carrying a gene for a highly toxic protein, such as DT-A, is the unwanted killing of the producer cell due to the expression of DT-A from the viral expression vector. The expression of DT-A from the lentiviral expression vector precluded the assembly of viral particles in vitro. However, by making a human cell line resistant to DT-A, we were able to overcome this obstacle and produce high titer DT-A expressing lentiviral particles.

To make a human cell line resistant to DT-A, we introduced a mutant human Elongation Factor 2 (EF2) gene into a human cell line using a retroviral vector as discussed in the Examples. Because the human cell line retains two copies of the endogenous EF2 gene, we did not know whether this mutagenesis strategy would successfully yield a DT-A resistant cell line. After eventually obtaining about 100 clones through this cloning strategy only 5 of them demonstrated DT-A resistance, and even then, the degree of DT-A resistance varied greatly among the different clones.

The nucleic acid sequence of the human EF2 gene is known and set forth at ACCESSION NM_{—}001961; VERSION NM_{—}001961.3 GI:83656775. The amino acid sequence of human EF2 is set forth below:

(SEQ ID NO. 17)
MVNFTVDQIRAIMDKKANIRNMSVIAHVDHGKSTLTDSLVCKAGIIASA
RAGETRFTDTRKDEQERCITIKSTAISLFYELSENDNFIKQSKDGAGFL
INLIDSPGHVDFSSEVTAALRVTDGALVVVDCVSGVCVQTETVLRQAIA
ERIKPVLMMNKMDRALLELQLEPEELYQTFQRIVENVNVIISTYGEGES
GPMGNIMIDPVLGTVGFGSGLHGWAFTLKQFAEMYVAKFAAKGEGQLGP
AERAKKVEDMMKKLWGDRYFDPANGKFSKSATSPEGKKLPRTFCQLILD
PIFKVFDAIMNFKKEETAKLIEKLDIKLDSEDKDKEGKPLLKAVMRRWL
PAGDALLQMITIHLPSPVTAQKYRCELLYEGPPDDEAAMGIKSCDPKGP
LMMYISKMVPTSDKGRFYAFGRVFSGLVSTGLKVRIMGPNYTPGKKEDL
YLKPIQRTILMMGRYVEPIEDVPCGNIVGLVGVDQFLVKTGTITTFEHA
HNMRVMKFSVSPVVRVAVEAKNPADLPKLVEGLKRLAKSDPMVQCIIEE
SGEHIIAGAGELHLEICLKDLEEDHACIPIKKSDPVVSYRETVSEESNV
LCLSKSPNKHNRLYMKARPFPDGLAEDIDKGEVSARQELKQRARYLAEK
YEWDVAEARKIWCFGPDGTGPNILTDITKGVQYLNEIKDSVVAGFQWAT
KEGALCEENMRGVRFDVHDVTLHADAIHRGGGQIIPTARRCLYASVLTA
QPRLMEPIYLVEIQCPEQVVGGIYGVLNRKRGHVFEESQVAGTPMFVVK
AYLPVNESFGFTADLRSNTGGQAFPQCVFDHWQILPGDPFDNSSRPSQV
VAETRKRKGLKEGIPALDNFLDKL

In one embodiment, the DT-A resistant human cell line has a mutation in the human EF2 gene that confers DT-A resistance to the human cell line. In another embodiment, the mutant EF2 has the same amino acid sequence as SEQ ID NO. 17 except for a substitution at position 717, such as a glycine to arginine substitution.

### 8. Formulations and Administration

The disclosure provides compositions comprising a NIRD lentiviral vector or NIRD lentiviral particles carrying a therapeutic gene, such as DT-A or TRAF6, as described herein. In certain embodiments, the compositions are suitable for pharmaceutical use and administration to patients. These compositions comprise a NIRD lentiviral vector or NIRD lentiviral particles carrying a therapeutic gene, such as DT-A or TRAF6, and a pharmaceutically acceptable excipient. The compositions may also contain other active compounds providing supplemental, additional, or enhanced therapeutic functions. The pharmaceutical compositions may also be included in a container, pack, or dispenser together with instructions for administration.

A pharmaceutical composition of the invention is formulated to be compatible with its intended route of administration. Methods to accomplish the administration are known to those of ordinary skill in the art. This includes, for example, injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically contemplated, by such means as depot injections or erodible implants.

Toxicity and therapeutic efficacy of the composition can be determined by standard pharmaceutical procedures in cell cultures or experimental animals, e.g., determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

Unless otherwise defined, all technical and scientific terms used herein have the same meaning as commonly understood by one of ordinary skill in the art. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

### EXAMPLES

All patents, patent applications, and published references cited herein are hereby incorporated by reference in their entirety. While this invention has been particularly shown and described with references to preferred embodiments thereof, it will be understood by those skilled in the art that various changes in form and details may be made therein without departing from the scope of the invention encompassed by the appended claims.

### Example 1

### Specificity of the Rev-Dependent Lentiviral Vectors in Mediating HIV-Dependent Gene Expression

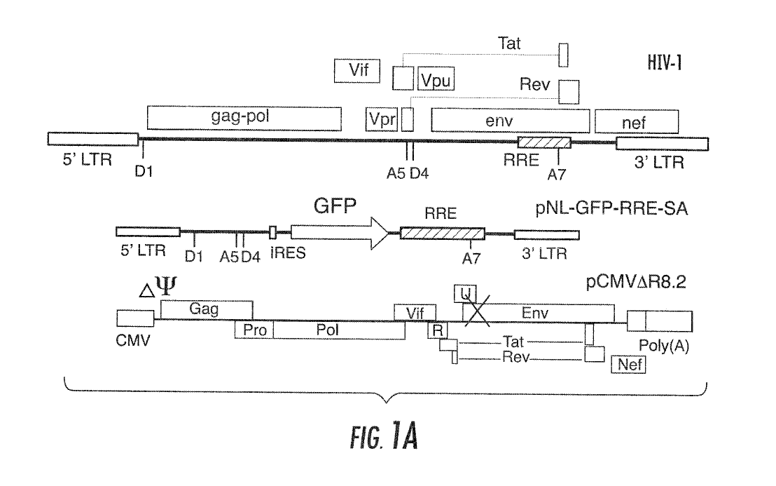

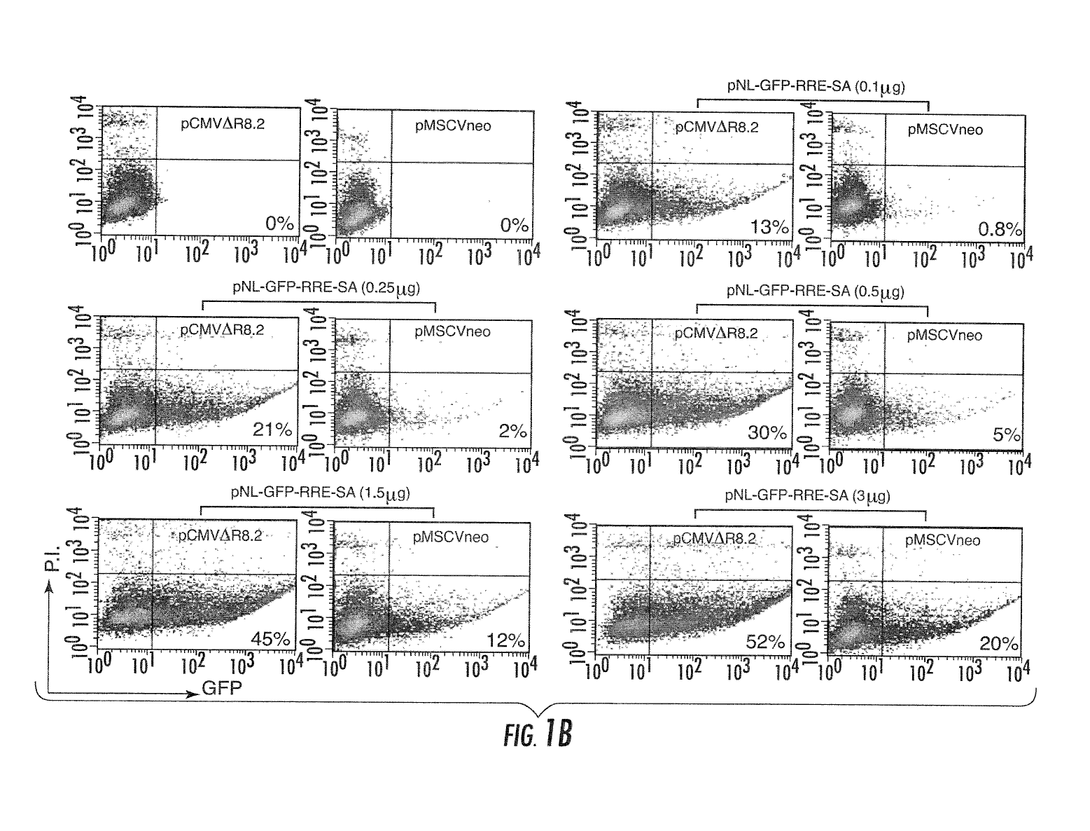

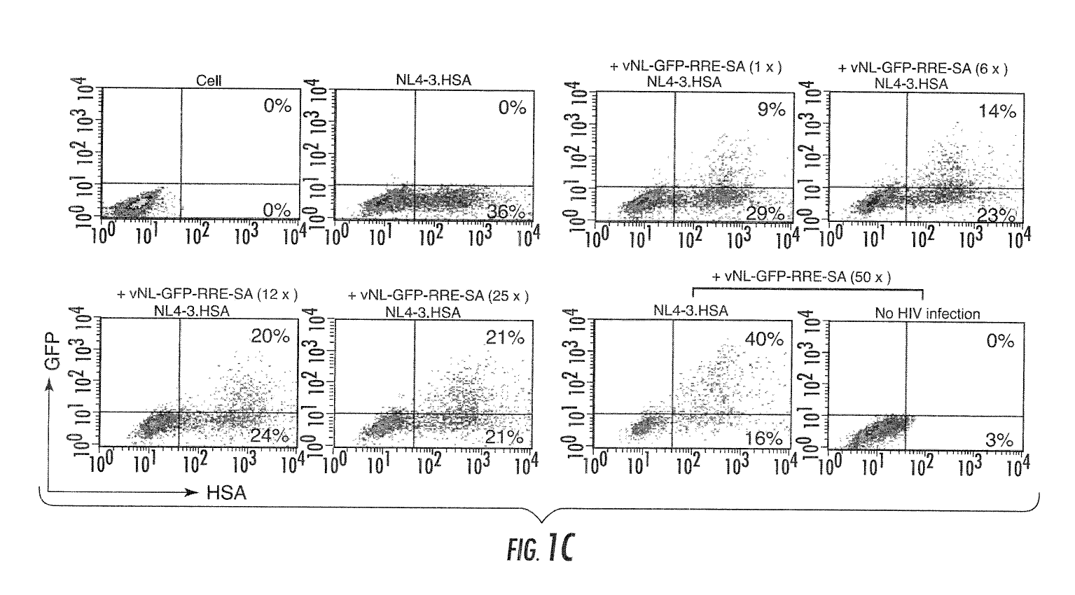

The Rev-dependent lentiviral vector was constructed based on the HIV-1 genome and has been described previously^{48,49}. As shown in FIG. 1A, we placed a reporter gene, the green fluorescent protein (GFP) gene, under the control of Rev by introducing multiple splicing sites and a Rev responsive element (RRE). This arrangement regulates GFP as a late gene and renders its expression specific to Rev.

To further demonstrate the specificity of this vector, we measured the expression of GFP mediated through viral infection or by cotransfection with a HIV-based helper plasmid, pCMVΔR8.2, which carries all viral genes and sequences except the packaging signal and the viral envelope⁵⁴ (FIG. 1A).

GFP expression was measured using flow cytometry. Briefly, one half to one million infected cells were removed from culture tubes and washed once with cold PBS, centrifuged for 5 minutes at 400×g and resuspended in 400 μl cold staining buffer (PBS plus 1% BSA). Nonspecific binding was blocked by adding 5 μl Rat IgG (10 mg/ml) (Jackson Laboratories Inc., Westgrove, Pa.). HIV-positive cells were stained with 2 μl of PE-labeled Rat Anti-Mouse CD24 (BioLegend, San Diego, Calif.). For isotype control staining, PE-labeled Rat IgG_{2b} (BioLegend, San Diego, Calif.) was used. Stained cells were incubated on ice for 30 minutes and then washed with cold PBS plus 1% BSA and resuspended in 500 μl of 1% paraformaldehyde for flow cytometry analysis on a FACSCalibur™ (BD Biosciences, San Jose, Calif.). Normally 10,000 to 20,000 cells were collected for analyses. Data analysis was performed using CellQuest™ (BD Biosciences, San Jose, Calif.) and FlowJo (Tree Star, San Carlos, Calif.).

When cotransfected with pCMVΔR8.2, pNL-GFP-RRE-SA expressed GFP in a dosage-dependent manner, generating GFP-positive cells from 13% (0.1 μg pNL-GFP-RRE-SA) to 52% (3 μg pNL-GFP-RRE-SA) (FIG. 1B). In contrast, when identically cotransfected with a control empty vector, pMSCVneo, which does not express HIV genes, pNL-GPF-RRE-SA expressed almost no GFP (0.8% GFP-positive cells) at the lowest dosage (0.1 μg). However, with the increasing amounts of pNL-GFP-RRE-SA, HIV-independent GFP expression was observed, and reached 20% of cells when 3 μg pNL-GFP-RRE-SA was used (FIG. 1B). Nevertheless, there was HIV-dependent enhancement of GFP expression in all dosages tested, and the enhancement was most dramatic at lower DNA dosages.

We also tested the specificity of the Rev-dependent vector when assembled into virion particles. Plasmids pNL-GFP-RRE-SA, pCMVΔR8.2, and pHCMV-G expressing the glycoprotein of vesicular stomatitis virus (VSV-G) were cotransfected into HEK293T cells using calcium phosphate.

Briefly, two million cells were cultured in a petri dish and cotransfected with 10 μg of pNL-GFP-RRE-SA, plus 7.5 μg of pCMVΔ8.2 and 2.5 μg of the VSV-G envelope construct pHCMV-G. Transfected cells were cultured overnight, and then the supernatant was removed and replaced with 10 ml fresh DMEM plus 10% heat-inactivated fetal bovine serum (FBS). Viruses were harvested at 48 and 72 hours and then concentrated by multiple rounds of concentration through an anion exchange column Sartobind® Q75 (Sartorius Stedium Biotech, Aubagne, France) and size-exclusion Vivaspin® 20 and 500 columns (Sartorius Stedium Biotech, Aubagne, France) using conditions as recommended by the manufacturer. Concentrated virus was divided into 50 μl aliquots and stored at −80° C.

Virion particles (vNL-GPF-RRE-SA) generated were harvested and concentrated to superinfect HIV-1-positive CEM-SS T cells, a human T cell line acquired from the NIH AIDS Research & Reference Reagent Program, NIAID and were cultured in RPMI 1640 medium supplemented with 10% heat-inactivated fetal bovine serum (FBS), penicillin (50 U/ml) and streptomycin (50 μg/ml) (Invitrogen, Carlsbad, Calif.). As shown in FIG. 1C, we observed dosage-dependent GFP expression exclusively in the HIV-1-positive cell population, whereas we did not observe any GFP expression in HIV-1-uninfected cells even with the highest multiplicity of infection (m.o.i.=10) by vNL-GFP-RRE-SA. This high stringency observed in viral infection was in great contrast to the cotransfection experiments, in which cells were overdosed with plasmid DNA and membrane disrupting agents. In cotransfection, even with 0.1 μg pNL-GFP-RRE-SA, each cell was roughly transfected with 10,000 molecules of pNL-GFP-RRE-SA, a condition that is significantly different from infection. Given that the Rev-dependent lentiviral vector is designed for gene delivery via infection, the specificity of the vector to express genes only in HIV-positive cells is reasonably high in infection conditions.

### Example 2

### Intracellular Cytotoxicity of DT-A and TRAF6 Expressed from the Rev-Dependent Vectors

To clone the DT-A chain and the human TRAF6 gene into the Rev-dependent vector, pNL-GFP-RRE-SA was used as the backbone^{48,49}. The codon-optimized DT-A chain carrying the start and stop codons was PCR amplified and cloned into pNL-GFP-REE-SA at the BamHI site. Specifically, DT-A was amplified from the plasmid template A-dmDT390biscFv (UCHT1)⁵⁰ using primers DT-BamHI-Start (5′CGCGGATCCATGGGTGCTGACGACGTCGTC3′) (SEQ ID NO. 1) and DT-BamHI-Stop (5′CGCGGATCCTTAGGCTTGAGCCATATACTCATA3′) (SEQ ID NO. 2). Cloning of DT-A was further confirmed by DNA sequence analysis. The packaging construct, pCMVΔ8.2, was kindly provided by Dr. Dider Trono.

The TRAF6 expressing plasmid, YFP-hTRAF6, was kindly provided by Dr. Liusheng He³⁹. TRAF6 gene was cloned into pNL-GFP-REE-SA at the BamHI site. Cloning of the TRAF6 gene was further confirmed by DNA sequence analysis.

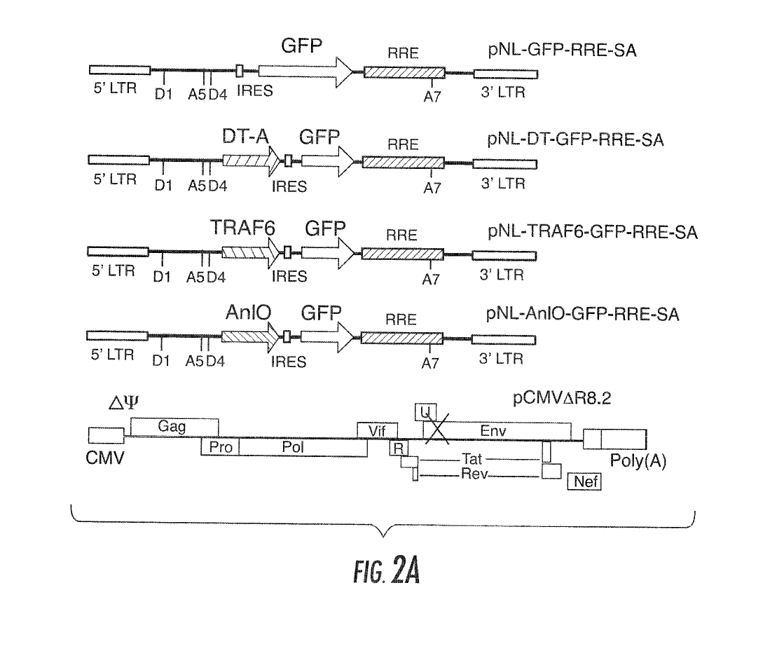

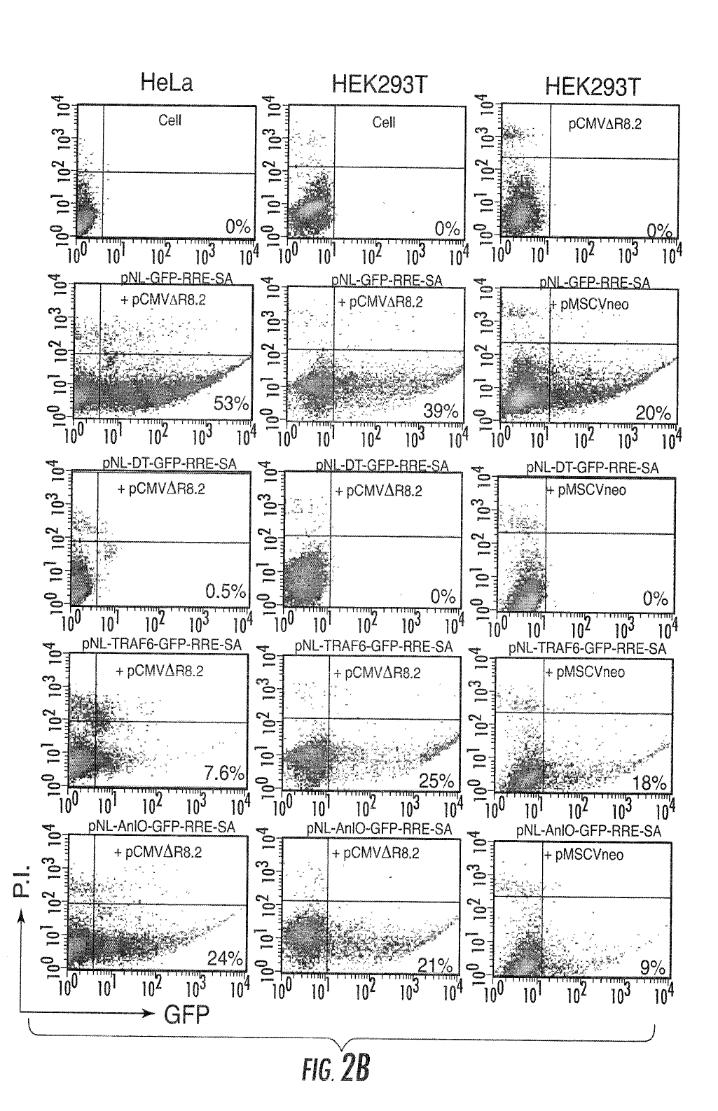

The pNL-GFP-RRE-SA vector contains an internal ribosome entry site (IRES) that allows the expression of two genes simultaneously (FIG. 2A). We demonstrated previously that the IRES permitted the use of GFP as a convenient indicator for the measurement of cell killing, since the accumulation of GFP inside the cell is prevented by co-expressed toxins. Thus, diminished GFP expression directly correlates with the cytotoxicity of the toxins²⁵.

We also used cotransfected HeLa and HEK293 T cells as model systems to compare Rev-dependent killing of cells by DT-A and TRAF6. Cells were cotransfected with the HIV-1 helper construct, pCMVΔR8.2, and either pNL-DT-GFP-RRE-SA, pNL-TRAF6-GFP-RRE-SA, or the control GFP vector, pNL-GFP-RRE-SA (FIG. 2B). The degree of cell killing from toxin expression was measured by comparing GFP expression in these parallel cotransfection experiments. As mentioned above, the reduction in the GFP-positive population was used as an indicator of toxin-mediated cell killing.

As shown in FIG. 2B, HeLa cells cotransfected with pCMVΔ8.2 plus the GFP vector pNL-GFP-RRE-SA generated 53% GFP-positive cells, whereas HeLa cells cotransfected with pCMVΔR8.2 plus the DT-A vector pNL-DT-GFP-RRE-SA generated almost no GFP-positive cells (0.5%). Similar results were also observed in cotransfected HEK293T cells, among which approximately 39% were GFP-positive when cotransfected with pCMCΔR8.2 plus pNL-GFP-RRE-SA, but none were GFP-positive when cotransfected with pCMVΔR8.2 plus pNL-DT-GFP-RRE-SA. These results are consistent with the universal killing of cells by DT-A at a single-molecule level³¹. In contrast, the TRAF6 vector generated 7.6% low-intensity GFP HeLa cells, suggesting that low-level expression of TRAF6 is tolerated to a certain extent (FIG. 2B). Indeed, as much as 25% GFP-positive cells were obtained in TRAF6 cotransfected HEK293T cells. These results also indicate that TRAF6 killing is cell-type dependent. HeLa cells appear to be more subject to TRAF6-induced apoptosis than HEK293T cells.

We also compared DT-A and TRAF6 with Anthrolysin 0 (AlnO), the first bacterial toxin tested in the Rev-dependent vector²⁵. It was apparent that DT-A and TRAF6 were more potent than AlnO in HeLa cells, but the killing by TRAF6 and AlnO was comparable in HEK293T cells (FIG. 2B). The different sensitivity of the two transformed cell lines, HeLa and HEK293T cells, to TRAF6-induction of apoptosis, is not currently understood. It may be related to the different levels of Rev expressed in these cells. Alternatively, it is also possible that certain cellular cofactors involved in the TRAF6-mediated apoptosis are differently expressed in these two cells. Nevertheless, these variations may not be an issue for in vivo targeting since most of the HIV-infected cells are non-transformed primary cells that should be vulnerable to apoptosis induction.

These Rev-dependent vectors were also cotransfected with an empty vector, pMSCVneo, instead of pCMVΔR8.2. Similar results were observed from the background toxin expression in the absence of HIV-1 proteins, demonstrating again that expression of these suicide genes can lead to the killing of GFP-positive cells.

### Example 3

### Mutagenesis of DT-A and the Construction of Rev-Dependent Lentiviral Vectors Carrying DT-A Mutants

When introduced into human cells, DT inactivates elongation factor 2 (EF-2) by ADP ribosylation and inhibits protein translation, which triggers apoptosis. DT is extremely toxic, and a single molecule can kill a cell³¹. This extreme toxicity is attractive for killing target cells with minimal toxin expression. In the meantime, it poses a significant problem in that any unexpected, non-specific expression in non-target cells would not be tolerated. DT-A mutants with reduced toxicity ranging from 30% to 0.2% have been successfully used previously^{55,56}.

We took advantage of these previous findings and generated a panel of DT-A chain mutants and tested their toxicity in cotransfected HEK293T cells. DT-A(E148S) has a single substitution of glutamic acid at codon 148 with serine, whereas DT-A(E148D) has a single substitution of the same amino acid with aspartic acid. Both mutants have been described previously⁵⁵. DT-A(176) was the mutation originally described in a DT-A chain mutant, tox176⁵⁶. The mutation was identified as a replacement of the glycine at codon 128 with aspartic acid.

The DT-A(E148D), DT-A(E148S), and DT-A(176) mutants were generated using a QuickChange® Site-Directed Mutagenesis Kit (Stratagene, La Jolla, Calif.) as recommended by the manufacturer.

The PCR primers used for generating DT-A(E148D) were

5′E148D
(SEQ ID NO. 3)
(5′GCTGAGGGTTCTTCTAGCGTTGATTATATTAATAACTGGGAACAG
3′);
and
3′E148D
(SEQ ID NO. 4)
(5′CTGTTCCCAGTTATTAATATAATCAACGCTAGAAGAACCCTCAGC
3′).

The primers for generating DT-A(E148S) were:

5′E148S
(SEQ ID NO. 5)
(5′GCTGAGGGTTCTTCTAGCGTTTCCTATATTAATAACTGGGAACAG
3′);
and
3′E148S
(SEQ ID NO. 6)
(5′CTGTTCCCAGTTATTAATATAGGAAACGCTAGAAGAACCCTCAGC
3′).

The PCR primers for generating DT-A(176) were 5′G128D(5′GAAGAGTTCATCAAGAGATTCGATGACGGTGCTTCCAGAGTCGTC3′) (SEQ ID NO. 7) and 3′G128D (5′GACGACTCTGGAAGCACCGTCATCGAATCTCTTGATGAACTCTTC3′) (SEQ ID NO. 8). All mutants were confirmed by sequence analysis.

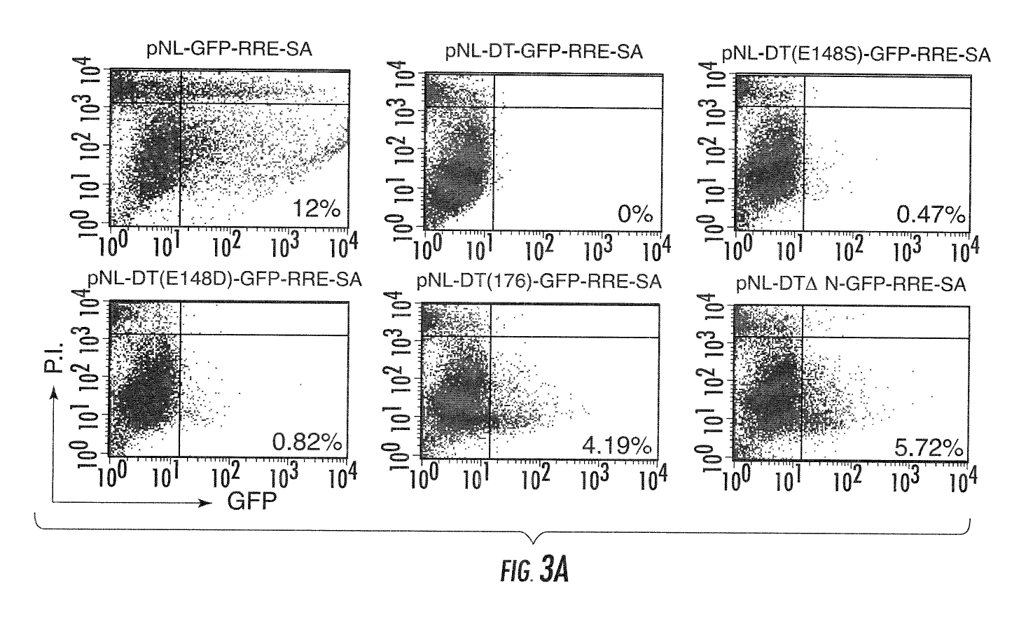

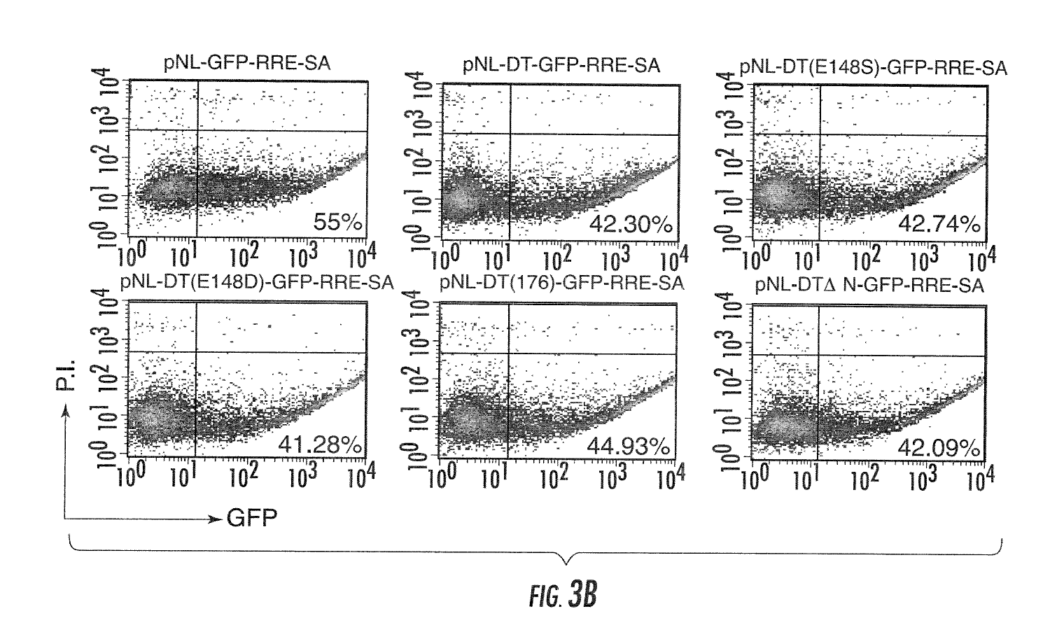

DT-AΔN was a new mutant generated in our own laboratory for the first time by removing the first 14 amino acids at the N-terminus of the DT-A chain. All of these DT-A mutants were cloned into the Rev-dependent vector, pNL-GFP-RRE-SA. When cotransfected with the helper plasmid, pCMVΔR8.2, into HEK293T cells, the Rev-dependent vectors carrying these DT mutants generated different percentages of GFP-positive cells in comparison with the wild-type DT-A. As shown in FIG. 3, while wild-type DT-A generated 0% GFP-positive cells, DT (E148S) generated 0.47%, DT (E148D) generated 0.82%, DT176 generated 4.19%, and DTΔN generated 5.72% of GFP-positive cells, respectively. The DTΔN-GFP is the least toxic of all the mutants and permitted the accumulation of low-level GFP in cells.

We also similarly cotransfected these plasmids into a DT-resistant, EF-2 mutant cell line, 5H7, which we constructed (see below). We observed a drastic increase in the GFP-positive cells, which were approximately 70-80% of those generated by the control vector pNL-GFP-RRE-SA (FIG. 3B). These data confirmed that the reductions in GFP percentages observed in HEK293T cells were related to EF-2 cytotoxicities of DT-A.

### Example 4

### Construction of DT-A-Resistant Cell Lines

The extreme toxicity of DT-A causes a problem in lentiviral production. Cotransfected HEK293T cells would be killed rapidly without being able to generate lentiviral particles carrying the DT-A gene. To solve this problem, we took advantage of a previous observation that a mutant hamster cell line carrying a single point mutation in the EF-2 gene confers resistance to the DT-A chain⁵⁷. The mutation was mapped to codon 717. We cloned the human EF-2 gene by PCR amplification and subsequently introduced a single point mutation (from G to A in the first nucleotide of codon 717) into EF-2.

Specifically, the human EF2 gene was cloned by RT-PCR amplification of total RNA extracted from HEK293T cells. The primers used for PCR were EF2-EcoRI (5′CCGGAATTCATGGTGAACTTCACGGTAGAC3′) (SEQ ID NO. 9) and EF2-XhoI (5′CCGCTCGAGCTACAATTTGTCCAGGAAGTTG3′) (SEQ ID NO. 10). The PCR product was digested with EcoRI and XhoI and subsequently inserted into pET17b at the EcoRI and XhoI site. Mutagenesis of the human EF2 gene was achieved by site-directed mutagenesis of codon 717 (G717R, GGA to CGA) using a Site-Directed Mutagenesis Kit (Stratagene, La Jolla, Calif.) and the primers 5′CACGCCGACGCCATCCACCGC CGAGGGGGCCAGATCATCCCC3′ (SEQ ID NO. 11) and 5′GGGGATGATCTGGCCCCCTCGGCGGTGGATGGCGTCGGCGTG3′(SEQ ID NO. 12). The human EF2 mutant was subcloned into the retroviral vector pMSCVneo (Clontech, Mountain View, Calif.) at the EcoRI and XhoI sites and subsequently transfected into the RetroPack™ PT67 cells (Clontech, Mountain View, Calif.) to be assembled into infectious viral particles. HEK293T cells were transduced with the viral particles and grown in 1 mg/ml Geneticin (Invitrogen, Carlsbad, Calif.).

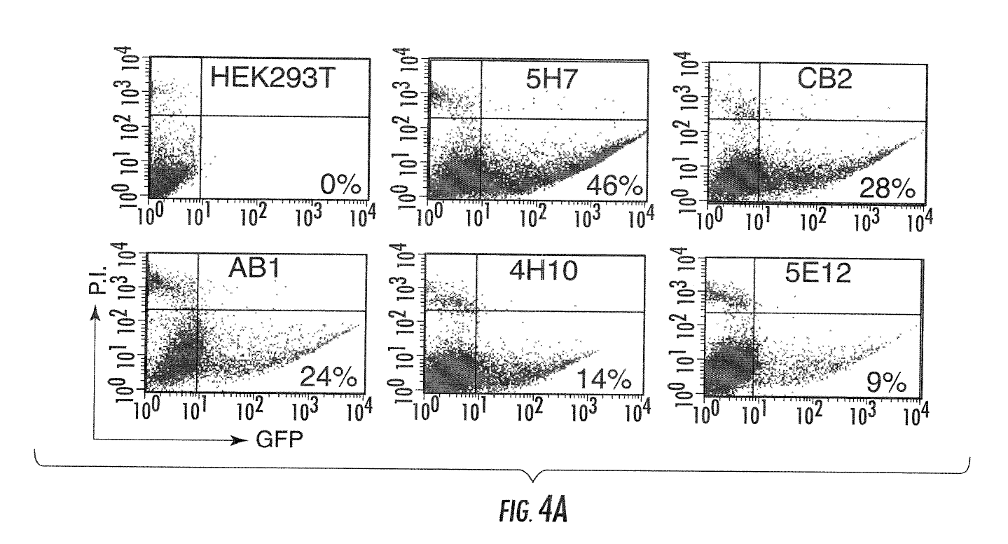

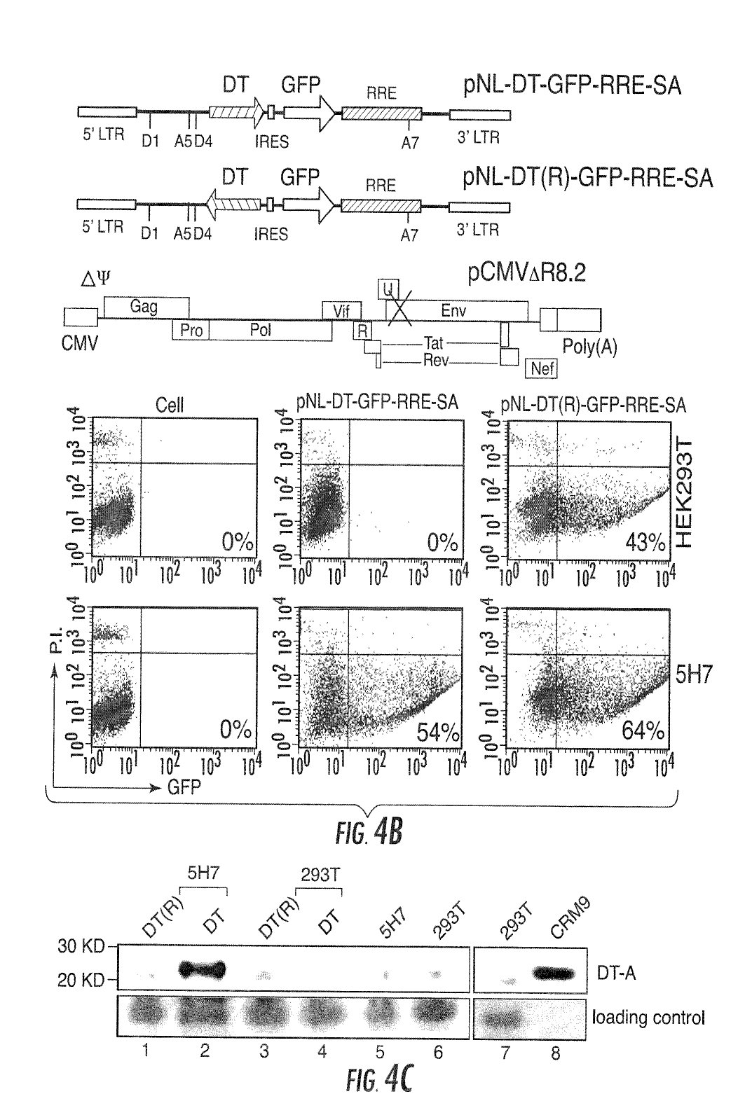

The mutant EF-2 was then introduced into human HEK293T cells by a retroviral vector for stable transduction. Cells were screened for mutant EF-2. Originally, we obtained about 100 clones, and 5 of them turned GFP-positive when cotransfected with the DT-A-containing lentiviral vector pNL-DT-GFP-RRE-SA plus the helper vector pCMVΔR8.2 (FIG. 4A). These cell clones were named 5H7, CB2, AB1, 4H10 and 5E12 and further tested for DT-A resistance. As shown in FIG. 4A, while the parental HEK293T cells generated 0% GFP-positive cells after cotransfection with pNL-DT-GFP-RRE-SA plus pCMVΔR8.2, these clones generated different percentages of GFP-positive cells: 46% in 5H7, 28% in CB2, 24% in AB1, 14% in 4H10, and 9% in 5E12, respectively. Clone 5H7 demonstrated the highest resistance to DT-A-mediated killing, and thus was selected as the DT-A resistant cell line for continuous culturing. To more accurately measure the degree of resistance of 5H7 to DT-A-mediated killing, both HEK293T cells and 5H7 cells were cotransfected identically with pCMVΔR8.2 plus pNL-DT-GFP-RRE-SA. As a control, cells were also cotransfected with pCMVΔR8.2 plus pNL-DT(R)-GFP-RRE-SA, a construct with DT-A cloned in the reverse orientation to prevent DT-A expression. As shown in FIG. 4B, in HEK293T cells, cotransfection with pNL-DT-GFP-RRE-SA generated 0% GFP-positive cells, whereas cotransfection with pNL-DT(R)-GFP-RRE-SA generated 43% GFP-positive cells. These results demonstrated 100% killing of HEK293T cells by DT-A. In contrast, in 5H7 cells, cotransfection with pNL-DT-GFP-RRE-SA generated 54% GFP-positive cells, whereas cotransfection with pNL-DT(R)-GFP-RRE-SA generated 64% GFP-positive cells. These results suggest that over 80% of the 5H7 cells survived the toxin. Some 5H7 cells can even tolerate high levels of DT-A expression, judging from the high levels of GFP expression observed (FIG. 4B).

We further confirmed intracellular expression of DT-A in 5H7 cells by Western blot using a monoclonal antibody against DT-A, which only detected a protein with the size of DT-A in 5H7 cells but not in HEK293T cells (FIG. 4C). Briefly, proteins in cell lysates from cotransfection were resolved on 4-20% SDS-polyacrylamide gel and electroblotted onto 0.2 μm nitrocellulose membrane. A 1:1000 dilution of a monoclonal antibody against DT (Meridian Life Science, Inc., Saco, Me.) or a goat polyclonal antibody against luciferase (Promega, Madison, Wis.) was incubated with the membrane, followed by a secondary goat antimouse antiserum (1:2000) or a rabbit polyclonal antigoat antibody (1:2000) conjugated with peroxidase (KPL, Gaithersburg, Md.). Chemiluminescence was captured on a cooled CCD camera using chemiluminescent SuperSignal® West Dura substrate (Pierce, Rockford, Ill.). The successful establishment of the DT-resistant 5H7 cells allowed us to assemble viral particles from the Rev-dependent DT-A vector.

### Example 5

### Construction of Non-Integrating Rev-Dependent (NIRD) Lentiviral Vector Carrying DT-A Chain and Human TRAF6

HIV Rev-regulated expression from the Rev-dependent lentiviral vector permitted selective expression of DT-A and TRAF6 in HIV-positive cells. However, the vector can enter both HIV-positive and -negative cells, and subsequently becomes integrated. Although we did not observe reporter gene expression in uninfected cells using GFP as a marker²⁵, any permanent integration of a toxin gene into the human genome poses a threat. Especially if the integration occurs at a transcriptionally active site, high-level gene expression may ensue and eventually trigger the Rev-independent protein synthesis and the subsequent death of an uninfected cell. To alleviate this potential risk, we decided to construct a NIRD lentiviral vector to deliver DT-A and TRAF6 as an episomal vector. Previously, we observed that a non-integrating HIV mutant, D116N, can transcribe from a DNA population as large as an integrating wild-type virus⁴⁴. However, each non-integrating DNA template is less active and expresses genes at a level approximately 10% that of an integrated proviral DNA⁴¹⁻⁴³. Additionally, we and others have also shown that D116N can express Rev-dependent late genes in the presence of high levels of Rev⁴² or the wild-type HIV-1⁵⁸. Thus, we constructed the NIRD vector based on D116N, and a single point mutation (from Asp to Asn) was introduced into pCMVΔR8.2 at the integrase amino acid 116 within the D(35)E functional motif. This single point mutation has been shown to completely abolish viral integrase activity without affecting other known viral functions such as reverse transcription and nuclear targeting

To demonstrate Rev-dependent expression of the NIRD vector in HIV-positive cells, we also cloned a luciferase reporter into the Rev-dependent vector and cotransfected this construct with pCMVΔR8.2(D116N) and the VSV-G envelope construct pHCMV-G, resulting in the production of a luciferase NIRD virion particle, vNL-Luc-RRE-SA(D116N). As a control, a similar integrating version of the viral particle, vNL-Luc-RRE-SA, was also assembled. These viruses were subsequently used to superinfect an HIV-1-positive Jurkat cell line, J1.1⁵¹, or to infect the HIV-negative, parental Jurkat cells as a control. The J1.1 cell line was acquired from the NIH AIDS Research & Reference Reagent Program, NIAID and was cultured in RPMI 1640 medium supplemented with 10% heat-inactivated fetal bovine serum (FBS), penicillin (50 U/ml) and streptomycin (50 μg/ml) (Invitrogen, Carlsbad, Calif.).

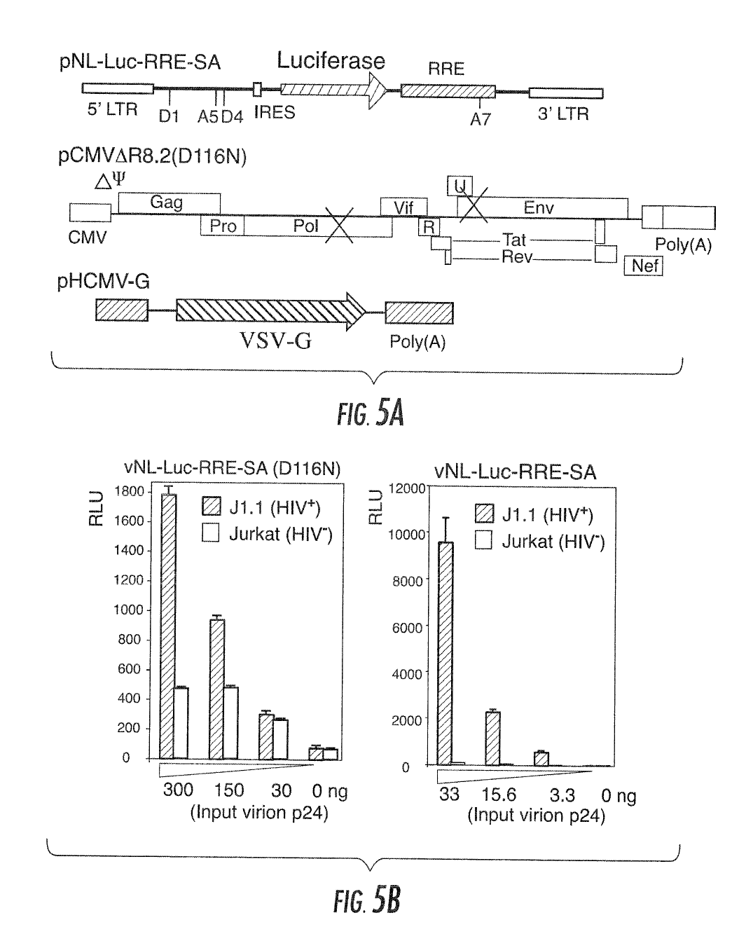

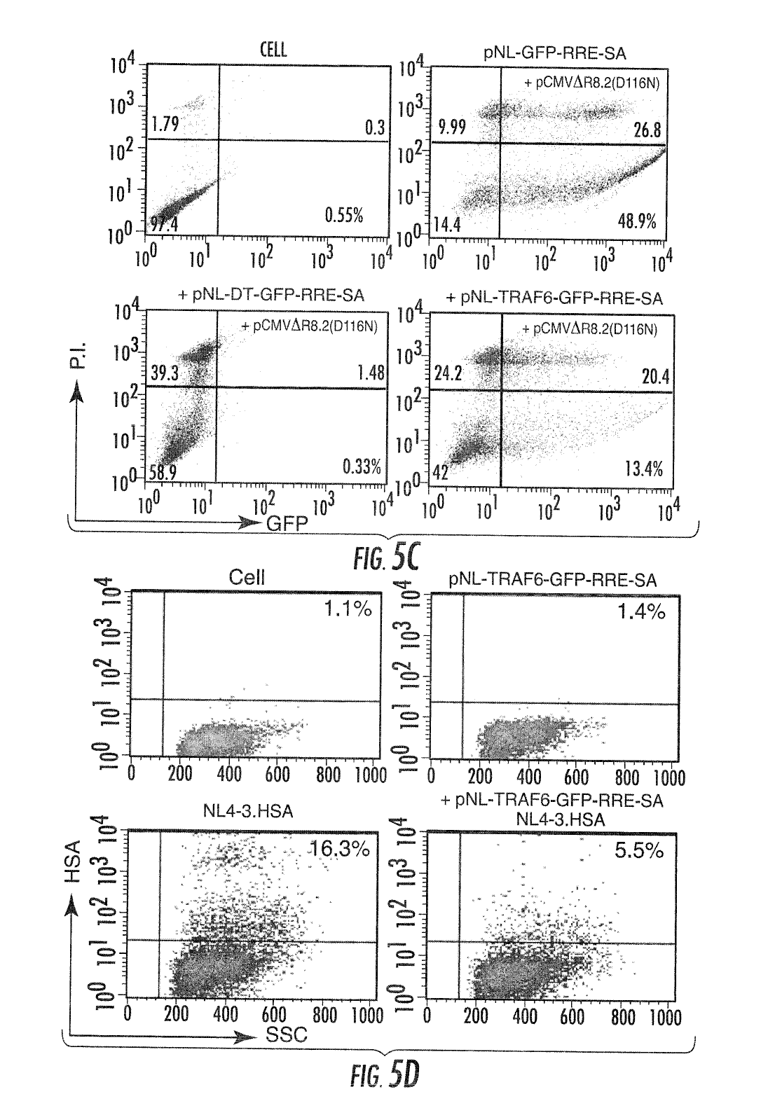

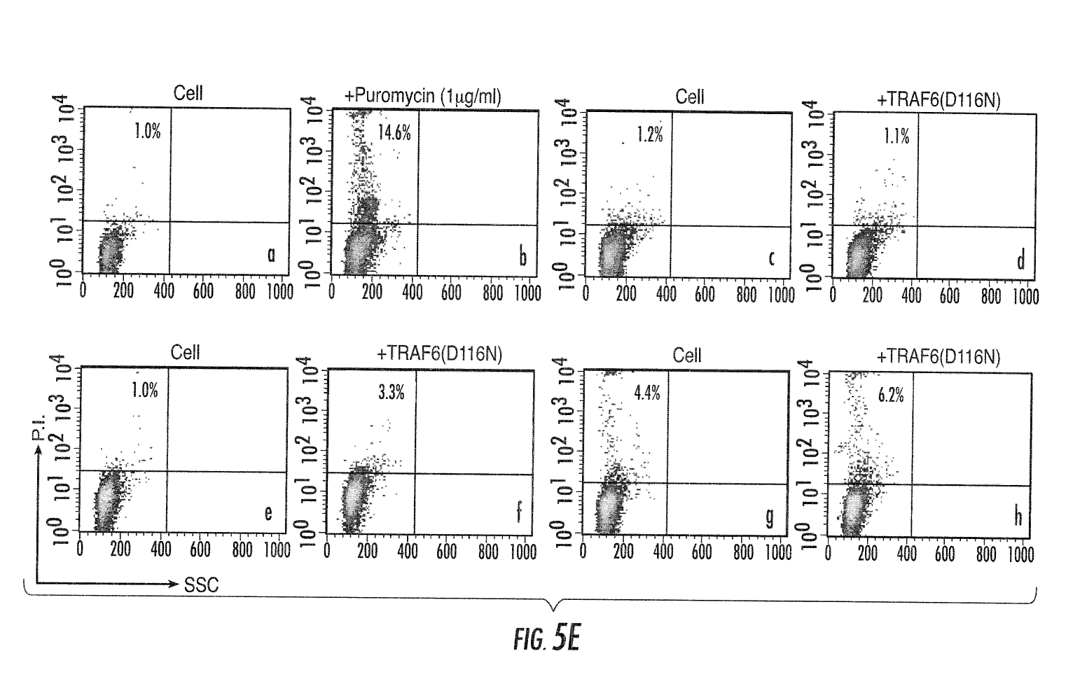

As shown in FIG. 5B, we detected dosage-dependent luciferase expression in J1.1 cells superinfected with vNL-Luc-RRE-SA(D116N) or vNL-Luc-RRE-SA, indicating that the NIRD vector was capable of mediating HIV-dependent gene expression. However, the expression level was much lower than that of the integrating vector. The background luciferase readings in HIV-negative Jurkat cells were likely derived from residual luciferase that could be present in the viral preparation and be subsequently introduced into cells during infection.

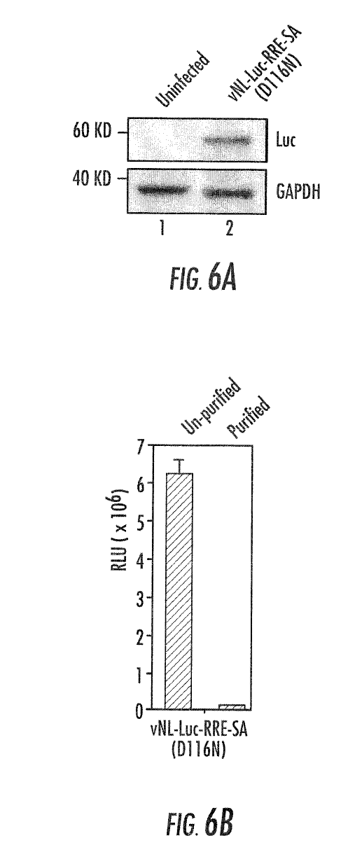

Indeed, a Western blot analysis of Jurkat cells immediately after infection (2 hours) detected the presence of the luciferase protein (FIG. 6A). Nevertheless, this background luciferase activity can be drastically reduced by purification of virion particles through anion exchange and size-exclusion columns (FIG. 6B).

Using lentiviral vector to deliver toxin genes faces potential problems of non-specific killing, one of which could be originated directly from toxin contamination of virion particles. It is well-known that lentiviral particles are normally contaminated with materials such as plasmid DNAs and non-viral proteins from producer cells during viral assembly. Nevertheless, we found that these non-viral proteins, such as the luciferase protein detected in the viral preparation (FIGS. 6A and 6B), can be drastically reduced through column purification (FIG. 6B). The problem of toxin contamination resembles the situation of plasmid DNA contamination of lentiviral particles. The plasmid DNA that contains cytopathic viral genes can usually be reduced by Benzonase treatment of virion particles to a minimal DNA level acceptable for clinical applications. In the case of toxin contamination, extensive virion purifications are likely required for reducing toxins to a low level that would not trigger non-specific killing of non-target cells.

To further confirm the killing of cells by DT-A and TRAF6, components of the NIRD constructs were cotransfected into HeLa cells. As shown in FIG. 5C, in cells cotransfected with pCMVΔR8.2(D116N) plus pNL-GFP-RRE-SA, we observed 48.9% GFP-positive cells, whereas in cells cotransfected with pCMVΔR8.2(D116N) plus pNL-DT-GFP-RRE-SA, we observed almost no GFP-positive cells (0.33%), demonstrating effective killing mediated by DT-A. We also observed killing of cells by TRAF6 at a lower efficiency, resulting in 13.4% GFP-positive cells (FIG. 5C).

### Example 6

### Production of NIRD Viral Particles Carrying DT-A and Human TRAF6 to Target HIV-Positive Cells

Given the demonstrated ability of the NIRD vectors described above, and the successful development of the DT-resistant 5H7 cell, we assembled the first NIRD viral particle carrying the DT-A gene. The NIRD particle carrying TRAF6 was also assembled in HEK293T cells due to the low toxicity of TRAF6 to this particular cell line (FIG. 2B). As shown in Table 1, following cotransfection of components of the NIRD vectors into 5H7 and HEK293T cells, viral particles were harvested at day 2 and day 3 post transfection, and comparable levels of viral production were obtained from the NIRD vectors and their integrating counterparts.

TABLE 1
Production of Integrating and NIRD Viral Particles carrying DT-A and TRAF6*

| | | p24 level at 48 hours | p24 level at 72 hours |
|---|---|---|---|
| DNA construct | Producer cell | post cotransfection | post cotransfection |
| pNL-TRAF6-GFP-RRE-SA | | | |
| pCMVΔR8.2 | HEK293T | 1095 ng/ml | 1659 ng/ml |
| pHCMV-G | | | |
| pNL-TRAF6-GFP-RRE-SA | | | |
| pCMVΔR8.2(D116N) | HEK293T | 870 ng/ml | 2059 ng/ml |
| pHCMV-G | | | |
| pNL-DTΔN-GFP-RRE-SA | | | |
| pCMVΔR8.2 | 5H7 | 109 ng/ml | 102 ng/ml |
| pHCMV-G | | | |
| pNL-DTΔN-GFP-RRE-SA | | | |
| pCMVΔR8.2(D116N) | 5H7 | 38 ng/ml | 60 ng/ml |
| pHCMV-G | | | |

*DNA constructs were cotransfected into producer cells cultured in 10 cm petri dish as described in Materials and Methods. Viral supernatant was harvested at 48 and 72 hours post infection, and levels of p24 were measured by ELISA.

The TRAF6 NIRD vector produced a p24 level of 870 ng/ml at day 2, whereas the DTΔN NIRD vector reached a p24 level of 60 ng/ml in 3 days. These viruses were concentrated 1000- to 2000-fold through an anion exchange column and size-exclusion columns to approximately 100-3000 mg/ml, a dosage sufficient for studies to target HIV-positive cells.

Viral p24 level was determined using a p24 ELISA assay (Beckman Coulter, Miami, Fla.). The titer of vNL-GFP-RRE-SA was measured directly on an HIV-1-positive cell line, J1.1⁵¹ (provided by the NIH AIDS Research & Reference Reagent Program, NIAID, NIH), which was cultured in 50 ng/ml PMA (phorbol myristate acetate) to stimulate HIV-1 activity. GFP-positive J1.1 cells were enumerated on FACSCalibur™ (BD Biosciences, San Jose, Calif.). The titers of vNL-DTΔN-GFP-RRE-SA and vNL-TRAF6-GFP-RRE-SA cannot be measured directly due to their cytolytic activity, and thus were estimated based on the p24 levels, using the titer of vNL-GFP-RRE-SA as a reference.

To test whether the assembled NIRD particles are capable of killing HIV-1-positive cells, human peripheral blood mononuclear cells (PBMC) were purified by centrifugation of blood cells on lymphocyte separation medium (Mediatech, Inc., Manassas, Va.) for 20 minutes at 400×g. Cells were washed twice with PBS buffer and resuspended into fresh RPMI 1640 medium supplemented with 10% FBS and cultured at 1×10⁶/ml. The lymphocyte subpopulation was infected with a replication-competent T-tropic virus, NL4-3.HSA.R+E+ (Vpr⁺, Env⁺), a clone with the murine heat-stable antigen CD24 (HSA) gene inserted into the nef region to facilitate the identification of HIV-1-positive cells by surface murine CD24 staining⁵². The HIV-1 strains, NL4-3.HSA.R+E−(VSV-G) and the replication-competent NL4-3.HSA.R+E+(“R” represents the Vpr gene and “E” represents the viral envelope gene)⁵² were provided by the NIH AIDS Research & Reference Reagent Program, NIAID, NIH. In both viruses, the murine heat-stable antigen CD24 (HSA) gene was inserted into the nef region that allows HIV-1-positive cells to be monitored by surface staining of HSA. Viruses were produced by transfection of HEK293T cells, using Lipofectamine 2000 (Invitrogen, Carlsbad, Calif.) as recommended by the manufacturer. HIV-1 titer was determined using an indicator cell line, Rev-CEM, as previously described⁴⁸.

Following HIV-1 infection for 24 hours, cells were superinfected with the NIRD particle, vNL-TRAF6-GFP-RRE-SA(D116N), and then continuously cultured for three days. For infection, cells were infected with HIV-1 for two hours at 37° C., and then washed twice with medium to remove unbound virus. Infected cells were resuspended into fresh medium. Superinfection was carried out by adding lentiviral particles directly to HIV-infected cells, followed by continuous culturing.

Two additional doses of the TRAF6 NIRD particles were then added at days 4 and 7 post HIV-1 infection. The spread of HIV-1 was monitored by HSA staining. As shown in FIG. 5E, HIV-1 replication resulted in the infection of 16.3% positive cells in 9 days. Superinfection with the TRAF6 NIRD particles reduced the HIV-positive cells to 5.5%, a 66% reduction in HIV-positive cells. Thus, the TRAF6 NIRD particles were shown to moderately reduce the population of HIV-positive cells (a 66% reduction) with three doses of superinfection. Given the transient nature of gene transcription in the absence of integration, it became apparent that the TRAF6 NIRD vector was not as effective as a previously tested, integrating Rev-dependent vector carrying anthrolysin O²⁵. However, the NIRD vector offers a significant safety advantage by reducing the possible risk of integration-mediated mutagenesis.

The selective reduction of HIV-positive cells did not result from possible non-specific killing by the TRAF6 NIRD particles. When HIV-uninfected cells were identically treated with the TRAF6 NIRD particles, we observed only a slight increase in cell death (from 4.4% to 6.6%, FIGS. 5E, g and h) in comparison with the untreated control, demonstrating that the TRAF6 NIRD particle resulted in about 2% non-specific killing of HIV-uninfected cells. This is significantly lower than the 66% reduction of HIV-positive cells. Based on these results, we calculated that approximately 97% of the killing mediated by TRAF6 NIRD particles was specific towards HIV-positive cells.

In the human body, cells that need to be targeted include infected macrophages and resting CD4 T cells, two of the major reservoirs of HIV-1^{3,6,10,11,15-17}. Macrophages are potentially the prime targets of the NIRD vector because these cells are long-lived and resistant to HIV-1-induced apoptosis. In addition, macrophages are relatively insensitive to antiretroviral drugs, and compartmentalized macrophages such as tissue and brain macrophages are hard to reach with drugs⁶³. These difficulties may potentially be compensated by the intracellular delivery of therapeutic genes through the NIRD particles. The non-integrated DNA delivered through the NIRD vector is known to persist for weeks and months in macrophages⁴³. This would permit low levels of HIV-dependent transcription to occur until a sufficient amount of toxins accumulated in macrophages to induce cell death. On the other hand, the lack of viral activity in resting CD4 T cells may pose a problem for the long-term efficacy of the NIRD vector against HIV-infected CD4 T cells. It may have to rely on transient stimulation of T cells with cytokines such as IL-2 and IFN-γ^{64,65} to permit transient gene expression to induce cell death.

### SEQUENCE LISTING

The instant application contains a Sequence Listing which has been submitted in ASCII format via EFS-Web and is hereby incorporated by reference in its entirety. Said ASCII copy, created on Oct. 28, 2010, is named Sequence_Listing_{—}0122.0003.txt and is 19 kilobytes in size.

### REFERENCES

The following references are cited in the application and provide general information on the field of the invention and provide assays and other details discussed in the application. The following references are incorporated herein by reference in their entirety.
1. Torres R A, Barr M. Impact of combination therapy for HIV infection on inpatient census. N Engl J Med 1997; 336: 1531-1532.
2. Ramratnam B, Mittler J E, Zhang L, Boden D, Hurley A, Fang F et al. The decay of the latent reservoir of replication-competent HIV-1 is inversely correlated with the extent of residual viral replication during prolonged anti-retroviral therapy. Nat Med 2000; 6: 82-85.
3. Zhu T, Muthui D, Holte S, Nickle D, Feng F, Brodie S et al. Evidence for human immunodeficiency virus type 1 replication in vivo in CD14(+) monocytes and its potential role as a source of virus in patients on highly active antiretroviral therapy. J Virol 2002; 76: 707-716.
4. Sedaghat A R, Siliciano J D, Brennan T P, Wilke C O, Siliciano R F. Limits on replenishment of the resting CD4+ T cell reservoir for HIV in patients on HAART. PLoS Pathog 2007; 3: e122.
5. Dybul M, Daucher M, Jensen M A, Hallahan C W, Chun T W, Belson M et al. Genetic characterization of rebounding human immunodeficiency virus type 1 in plasma during multiple interruptions of highly active antiretroviral therapy. J Virol 2003; 77: 3229-3237.
6. Finzi D, Hermankova M, Pierson T, Carruth L M, Buck C, Chaisson R E et al. Identification of a reservoir for HIV-1 in patients on highly active antiretroviral therapy [see comments]. Science 1997; 278: 1295-1300.
7. Chun T W, Stuyver L, Mizell S B, Ehler L A, Mican J A, Baseler M et al. Presence of an inducible HIV-1 latent reservoir during highly active antiretroviral therapy. Proc Natl Acad Sci USA 1997; 94: 13193-13197.
8. Wong J K, Hezareh M, Gunthard H F, Havlir D V, Ignacio C C, Spina C A et al. Recovery of replication-competent HIV despite prolonged suppression of plasma viremia [see comments]. Science 1997; 278: 1291-1295.
9. Furtado M R, Callaway D S, Phair J P, Kunstman K J, Stanton J L, Macken C A et al. Persistence of HIV-1 transcription in peripheral-blood mononuclear cells in patients receiving potent antiretroviral therapy. N Engl J Med 1999; 340: 1614-1622.
10. Lambotte O, Taoufik Y, de Goer M G, Walton C, Goujard C, Delfraissy J F. Detection of infectious HIV in circulating monocytes from patients on prolonged highly active antiretroviral therapy. J Acquir Immune Defic Syndr 2000; 23: 114-119.
11. Crowe S M, Sonza S. HIV-1 can be recovered from a variety of cells including peripheral blood monocytes of patients receiving highly active antiretroviral therapy: a further obstacle to eradication. J Leukoc Biol 2000; 68: 345-350.
12. Finzi D, Blankson J, Siliciano J D, Margolick J B, Chadwick K, Pierson T et al. Latent infection of CD4+ T cells provides a mechanism for lifelong persistence of HIV-1, even in patients on effective combination therapy. Nat Med 1999; 5: 512-517.
13. Koenig S, Gendelman H E, Orenstein J M, Dal Canto M C, Pezeshkpour G H, Yungbluth M et al. Detection of AIDS virus in macrophages in brain tissue from AIDS patients with encephalopathy. Science 1986; 233: 1089-1093.
14. Ramratnam B, Ribeiro R, He T, Chung C, Simon V, Vanderhoeven J et al. Intensification of antiretroviral therapy accelerates the decay of the HIV-1 latent reservoir and decreases, but does not eliminate, ongoing virus replication. J Acquir Immune Defic Syndr 2004; 35: 33-37.
15. Sonza S, Mutimer H P, Oelrichs R, Jardine D, Harvey K, Dunne A et al. Monocytes harbour replication-competent, non-latent HIV-1 in patients on highly active antiretroviral therapy. Aids 2001; 15: 17-22.
16. Igarashi T, Brown C R, Endo Y, Buckler-White A, Plishka R, Bischofberger N et al. Macrophage are the principal reservoir and sustain high virus loads in rhesus macaques after the depletion of CD4+ T cells by a highly pathogenic simian immunodeficiency virus/HIV type 1 chimera (SHIV): Implications for HIV-1 infections of humans. Proc Natl Acad Sci USA 2001; 98: 658-663.
17. Chomont N, El-Far M, Ancuta P, Trautmann L, Procopio F A, Yassine-Diab B et al. HIV reservoir size and persistence are driven by T cell survival and homeostatic proliferation. Nat Med 2009; 15: 893-900.

18, Aquaro S, Calio R, Balestra E, Bagnarelli P, Cenci A, Bertoli A et al. Clinical implications of HIV dynamics and drug resistance in macrophages. J Biol Regul Homeost Agents 1998; 12: 23-27.
19. Autran B, Carcelain G, Li T S, Blanc C, Mathez D, Tubiana R et al. Positive effects of combined antiretroviral therapy on CD4+ T cell homeostasis and function in advanced HIV disease. Science 1997; 277: 112-116.
20. Perelson A S, Essunger P, Cao Y, Vesanen M, Hurley A, Saksela K et al. Decay characteristics of HIV-1-infected compartments during combination therapy. Nature 1997; 387: 188-191.
21. Richman D D, Margolis D M, Delaney M, Greene W C, Hazuda D, Pomerantz R J. The challenge of finding a cure for HIV infection. Science 2009; 323: 1304-1307.
22. Rosenberg E S, Billingsley J M, Caliendo A M, Boswell S L, Sax P E, Kalams S A et al, Vigorous HIV-1-specific CD4+ T cell responses associated with control of viremia. Science 1997; 278: 1447-1450.
23. Hatano H, Vogel S, Yoder C, Metcalf J A, Dewar R, Davey R T, Jr. et al. Pre-HAART HIV burden approximates post-HAART viral levels following interruption of therapy in patients with sustained viral suppression. Aids 2000; 14: 1357-1363.
24. Davey R T, Jr., Bhat N, Yoder C, Chun T W, Metcalf J A, Dewar R et al. HIV-1 and T cell dynamics after interruption of highly active antiretroviral therapy (HAART) in patients with a history of sustained viral suppression. Proc Natl Acad Sci USA 1999; 96: 15109-15114.
25. Young J, Tang Z, Yu Q, Yu D, Wu Y. Selective killing of HIV-1-positive macrophages and T Cells by the Rev-dependent lentivirus carrying anthrolysin O from Bacillus anthracis. Retrovirology 2008; 5: 36.
26. Shannon J G, Ross C L, Koehler T M, Rest R F. Characterization of anthrolysin O, the Bacillus anthracis cholesterol-dependent cytolysin. Infection & Immunity 2003; 71: 3183-3189.
27. King W, Patel M D, Lobel L I, Goff S P, Nguyen-Huu M C. Insertion mutagenesis of embryonal carcinoma cells by retroviruses. Science 1985; 228: 554-558.
28. Li Z, Dullmann J, Schiedlmeier B, Schmidt M, von Kalle C, Meyer J et al. Murine leukemia induced by retroviral gene marking. Science 2002; 296: 497.
29. Greenfield L, Bjorn M J, Horn G, Fong D, Buck G A, Collier R J et al. Nucleotide sequence of the structural gene for diphtheria toxin carried by corynebacteriophage beta. Proc Natl Acad Sci USA 1983; 80: 6853-6857.
30. Kochi S K, Collier R J. DNA fragmentation and cytolysis in U937 cells treated with diphtheria toxin or other inhibitors of protein synthesis. Exp Cell Res 1993; 208: 296-302.
31. Yamaizumi M, Mekada E, Uchida T, Okada Y. One molecule of diphtheria toxin fragment A introduced into a cell can kill the cell. Cell 1978; 15: 245-250.
32. Kunitomi M, Takayama E, Suzuki S, Yasuda T, Tsutsui K, Nagaike K et al. Selective inhibition of hepatoma cells using diphtheria toxin A under the control of the promoter/enhancer region of the human alpha-fetoprotein gene. Jpn J Cancer Res 2000; 91: 343-350.
33. Kelley V E, Bacha P, Pankewycz O, Nichols J C, Murphy J R, Strom T B. Interleukin 2-diphtheria toxin fusion protein can abolish cell-mediated immunity in vivo. Proc Natl Acad Sci USA 1988; 85: 3980-3984.
34. Arora N, Masood R, Zheng T, Cai J, Smith D L, Gill P S. Vascular endothelial growth factor chimeric toxin is highly active against endothelial cells. Cancer Res 1999; 59: 183-188.
35. Kuzel T M, Rosen S T, Gordon L I, Winter J, Samuelson E, Kaul K et al. Phase I trial of the diphtheria toxin/interleukin-2 fusion protein DAB486IL-2: efficacy in mycosis fungoides and other non-Hodgkin's lymphomas. Leuk Lymphoma 1993; 11: 369-377.
36. Frankel A E, Powell B L, Hall P D, Case L D, Kreitman R J. Phase I trial of a novel diphtheria toxin/granulocyte macrophage colony-stimulating factor fusion protein (DT388GMCSF) for refractory or relapsed acute myeloid leukemia. Clin Cancer Res 2002; 8: 1004-1013.
37. Frankel A E, Surendranathan A, Black J H, White A, Ganjoo K, Cripe L D. Phase II clinical studies of denileukin diftitox diphtheria toxin fusion protein in patients with previously treated chronic lymphocytic leukemia. Cancer 2006; 106: 2158-2164.
38. Li Y, McCadden J, Ferrer F, Kruszewski M, Carducci M, Simons J et al. Prostate-specific expression of the diphtheria toxin A chain (DT-A): studies of inducibility and specificity of expression of prostate-specific antigen promoter-driven DT-A adenoviral-mediated gene transfer. Cancer Res 2002; 62: 2576-2582.
39. He L, Wu X, Siegel R, Lipsky P E. TRAF6 regulates cell fate decisions by inducing caspase 8-dependent apoptosis and the activation of NF-kappa B. J Biol Chem 2006.
40. Engelman A, Englund G, Orenstein J M, Martin M A, Craigie R. Multiple effects of mutations in human immunodeficiency virus type 1 integrase on viral replication. J Virol 1995; 69: 2729-2736.
41. Wu Y, Marsh J W. Selective transcription and modulation of resting T cell activity by preintegrated HIV DNA. Science 2001; 293: 1503-1506.
42. Wu Y, Marsh J W. Early transcription from nonintegrated DNA in human immunodeficiency virus infection. J Virol 2003; 77: 10376-10382.
43. Kelly J, Beddall M H, Yu D, Iyer S R, Marsh J W, Wu Y. Human macrophages support persistent transcription from unintegrated HIV-1 DNA. Virology 2008; 372: 300-312.
44. Iyer S, Yu D, Biancotto A, Margolis L, Wu Y. Measurement of HIV-1 preintegration transcription using the Rev-dependent Rev-CEM cell reveals a sizable transcribing DNA population comparable with proviral templates. Journal of Virology 2009.
45. Philippe S, Sarkis C, Barkats M, Mammeri H, Ladroue C, Petit C et al. Lentiviral vectors with a defective integrase allow efficient and sustained transgene expression in vitro and in vivo. Proc Natl Acad Sci USA 2006; 103: 17684-17689.
46. Vargas J. Jr.; Klotman M E, Cara A. Conditionally replicating lentiviral-hybrid episomal vectors for suicide gene therapy. Antiviral Res 2008; 80: 288-294.
47. Vargas J, Jr., Gusella G L, Najfeld V, Klotman M E, Cara A. Novel integrase-defective lentiviral episomal vectors for gene transfer. Hum Gene Ther 2004; 15: 361-372.
48. Wu Y, Beddall M H, Marsh J W. Rev-dependent indicator T cell line. Current HIV Research 2007; 5: 395-403.
49. Wu Y, Beddall M H, Marsh J W. Rev-dependent lentiviral expression vector. Retrovirology 2007; 4: 12.
50. Woo J H, Liu Y Y, Mathias A, Stavrou S, Wang Z, Thompson J et al. Gene optimization is necessary to express a bivalent anti-human anti-T cell immunotoxin in Pichia pastoris. Protein Expr Purif 2002; 25: 270-282.
51. Perez V L, Rowe T, Justement J S, Butera S T, June C H, Folks T M. An HIV-1-infected T cell clone defective in IL-2 production and Ca2+ mobilization after CD3 stimulation. Journal of Immunology 1991; 147: 3145-3148.
52. He J, Choe S, Walker R, Di Marzio P, Morgan D O, Landau N R. Human immunodeficiency virus type 1 viral protein R (Vpr) arrests cells in the G2 phase of the cell cycle by inhibiting p34cdc2 activity. J Virol 1995; 69: 6705-6711.
53. Foley G E, Lazarus H, Farber S, Uzman B G, Boone B A, McCarthy R E. Continuous Culture of Human Lymphoblasts from Peripheral Blood of a Child with Acute Leukemia. Cancer 1965; 18: 522-529.
54. Naldini L, Blomer U, Gallay P, Ory D, Mulligan R, Gage F H et al. In vivo gene delivery and stable transduction of nondividing cells by a lentiviral vector. Science 1996; 272: 263-267.
55. Fisher K S, Maxwell I H, Murphy J R, Collier J, Glode L M. Construction and expression of plasmids containing mutated diphtheria toxin A-chain-coding sequences. Infect Immun 1991; 59: 3562-3565.
56. Maxwell F, Maxwell I H, Glode L M. Cloning, sequence determination, and expression in transfected cells of the coding sequence for the tox 176 attenuated diphtheria toxin A chain. Mol Cell Biol 1987; 7: 1576-1579.
57. Kohno K, Uchida T. Highly frequent single amino acid substitution in mammalian elongation factor 2 (EF-2) results in expression of resistance to EF-2-ADP-ribosylating toxins. J Biol Chem 1987; 262: 12298-12305.
58. Gelderblom H C, Vatakis D N, Burke S A, Lawrie S D, Bristol G C, Levy D N. Viral complementation allows HIV-1 replication without integration. Retrovirology 2008.
59. Marsden M D, Zack J A. Eradication of HIV: current challenges and new directions. J Antimicrob Chemother 2009; 63: 7-10.
60. Chun T W, Davey R T, Jr., Engel D, Lane H C, Fauci A S. Re-emergence of HIV after stopping therapy [In Process Citation]. Nature 1999; 401: 874-875.
61. Gaur M, Leavitt A D. Mutations in the human immunodeficiency virus type I integrase D,D(35)E motif do not eliminate provirus formation. J Virol 1998; 72: 4678-4685.
62. Leavitt A D, Robles G, Alesandro N, Varmus H E. Human immunodeficiency virus type 1 integrase mutants retain in vitro integrase activity yet fail to integrate viral DNA efficiently during infection. J Virol 1996; 70: 721-728.
63. Aquaro S, Calio R, Balzarini J, Bellocchi M C, Garaci E, Perno C F. Macrophages and HIV infection: therapeutical approaches toward this strategic virus reservoir. Antiviral Res 2002; 55: 209-225.
64. Stellbrink H J, Hufert F T, Tenner-Racz K, Lauer J, Schneider C, Albrecht H et al. Kinetics of productive and latent HIV infection in lymphatic tissue and peripheral blood during triple-drug combination therapy with or without additional interleukin-2. Antivir Ther 1998; 3: 209-214.
65. Chun T W, Engel D, Mizell S B, Hallahan C W, Fischette M, Park S et al. Effect of interleukin-2 on the pool of latently infected, resting CD4+ T cells in HIV-1-infected patients receiving highly active anti-retroviral therapy [see comments]. Nat Med 1999; 5: 651-655.
66. Karin M, Lin A. NF-kappaB at the crossroads of life and death. Nat Immunol 2002; 3: 221-227.
67. Greene C, O'Neill L. Interleukin-1 receptor-associated kinase and TRAF-6 mediate the transcriptional regulation of interleukin-2 by interleukin-1 via NFkappaB but unlike interleukin-1 are unable to stabilise interleukin-2 mRNA. Biochim Biophys Acta 1999; 1451: 109-121.
68. Matsumura T, Degawa T, Takii T, Hayashi H, Okamoto T, Inoue J et al. TRAF6-NF-kappaB pathway is essential for interleukin-1-induced TLR2 expression and its functional response to TLR2 ligand in murine hepatocytes. Immunology 2003; 109: 127-136.
69. Goor R S, Pappenheimer A M, Jr. Studies on the mode of action of diphtheria toxin. 3. Site of toxin action in cell-free extracts. J Exp Med 1967; 126: 899-912.
70. Collier R J. Effect of diphtheria toxin on protein synthesis: inactivation of one of the transfer factors. J Mol Biol 1967; 25: 83-98.
71. Collier R J. Diphtheria toxin: mode of action and structure. Bacteriol Rev 1975; 39: 54-85.
72. Collier R J. Understanding the mode of action of diphtheria toxin: a perspective on progress during the 20th century. Toxicon 2001; 39: 1793-1803.
73. Potala S, Sahoo S K, Verma R S. Targeted therapy of cancer using diphtheria toxin-derived immunotoxins. Drug Discov Today 2008; 13: 807-815.
74. Neville D M, Jr., Scharff J, Hu H Z, Rigaut K, Shiloach J, Slingerland W et al. A new reagent for the induction of T-cell depletion, anti-CD3-CRM9. J Immunother Emphasis Tumor Immunol 1996; 19: 85-92.
75. Purcell, D. F. and Martin, M. A. Alternative splicing of human immunodeficiency virus type 1 mRNA modulates viral protein expression, replication, and infectivity. J. Virol. 1993; 67:6365-78.
76. Rana, T. M. and Jeang, K. T. Biochemical and functional interactions between HIV-1 Tat protein and TAR RNA. Arch. Biochem. Biophys. 1999; 365:175-185.
77. Pollard, V. W. and Malim, M. H. The HIV-1 Rev protein. Annu. Rev. Microbiol. 1998; 52:491-532.

What is claimed:

1. A lentiviral particle comprising:
a) a nucleic acid molecule comprising:
i) a promoter, wherein the activity of the promoter is dependent on the presence of a human immunodeficiency virus (HIV) Tat protein;
ii) at least one splice donor site and at least one splice acceptor site;
iii) a nucleotide sequence encoding human TRAF6 or diphtheria toxin A, wherein at least part of the nucleotide sequence is located in an intron between the at least one splice acceptor site and the at least one donor acceptor site; and
iv) a Rev Responsive Element (RRE) from a HIV,
wherein elements i)-iv) are operably linked;
b) a reverse transcriptase;
c) one or more lentiviral proteins selected from a matrix protein, a capsid protein, a nucleocapsid protein, Vif, Vpr, Vpu, Nef, and Tat; and
d) a mutant integrase, wherein the mutant integrase cannot integrate the nucleic acid molecule into a host cell genome.

2. The lentiviral particle of claim 1, wherein the lentiviral particle is an HIV particle.

3. The lentiviral particle of claim 1, wherein the reverse transcriptase is encoded by an HIV pol gene.

4. The lentiviral particle of claim 1, wherein the mutant integrase comprises a mutation at amino acid 116 of the integrase encoded by the HIV pol gene.

5. A method of producing the lentiviral particle of claim 1, comprising transfecting into a host cell under conditions permitting the production of the lentivirall particle:
a) a first vector comprising a nucleic acid molecule comprising:
i) a promoter, wherein the activity of the promoter is dependent on the presence of the human immunodeficiency virus (HIV) Tat protein;
ii) at least one splice donor site and at least one splice acceptor site;
iii) a first nucleotide sequence encoding human TRAF6 or diphtheria toxin A, wherein at least part of the first nucleotide sequence is located in an intron between the at least one splice acceptor site and the at least one donor acceptor site; and
iv) a Rev Responsive Element (RRE) from the HIV,
wherein elements i)-iv) are operably linked;
b) a second vector comprising a second nucleotide sequence comprising a lentiviral gag gene and a lentiviral pol gene, wherein the lentiviral pol gene encodes a mutant integrase and wherein the mutant integrase cannot integrate the nucleic acid molecule into the genome of the host cell; and
c) a third vector comprising a third nucleotide sequence encoding a viral envelope protein.

6. The method of claim 5, further comprising recovering the lentiviral particles produced by the host cell.

7. The method of claim 5, wherein the first nucleotide sequence encodes human TRAF6.

8. The method of claim 5, wherein the first nucleotide sequence encodes diphtheria toxin A and wherein the host cell comprises a mutant human EF2 gene that confers diphtheria toxin A resistance to the host cell.

9. The method of claim 8, wherein the mutant human EF2 gene comprises the amino acid sequence of SEQ ID NO. 17 except for a substitution at amino acid 717.

10. The method of claim 5, wherein the first nucleotide sequence encodes a mutant diphtheria toxin A, wherein the mutant diphtheria toxin A is less toxic than the wild type diphtheria toxin A.

11. The method of claim 5, wherein the lentiviral particle is an HIV particle.

12. The method of claim 5, wherein the second nucleotide sequence of the second vector further comprises one or more lentiviral genes selected from vif, vpr, vpu, vpx, tat, nef, and tat.

13. The method of claim 5, wherein the viral envelope protein is a vesicular stomatitis virus G protein.

14. A lentiviral particle produced according to the method of claim 5.

15. An isolated human host cell comprising a mutant human EF2 gene, wherein the mutant human EF2 gene comprises the amino acid sequence of SEQ ID NO. 17 except for a substitution at amino acid 717 and wherein the mutant human EF2 gene confers diphtheria toxin A resistance to the host cell.

16. The host cell of claim 15, further comprising a nucleic acid molecule comprising:
a) a promoter, wherein the activity of the promoter is dependent on the presence of the human immunodeficiency virus (HIV) Tat protein;
b) at least one splice donor site and at least one splice acceptor site;
c) a nucleotide sequence encoding diphtheria toxin A, wherein at least part of the nucleotide sequence is located in an intron between the at least one splice acceptor site and the at least one donor acceptor site; and
d) a Rev Responsive Element (RRE) from the HIV,
wherein elements a)-d) are operably linked.

17. A method of killing a cell infected with HIV, the method comprising contacting the cell with a lentiviral particle of claim 1.

18. An isolated nucleic acid molecule comprising:
a) a promoter, wherein the activity of the promoter is dependent on the presence of the human immunodeficiency virus (HIV) Tat protein;
b) at least one splice donor site and at least one splice acceptor site;
c) a nucleotide sequence encoding human TRAF6 or diphtheria toxin A, wherein at least part of the nucleotide sequence is located in an intron between the at least one splice acceptor site and the at least one donor acceptor site; and
d) a Rev Responsive Element (RRE) from the HIV,
wherein elements a)-d) are operably linked.

19. The nucleic acid molecule of claim 18, wherein the nucleotide sequence encodes a mutant diphtheria toxin A, wherein the mutant diphtheria toxin A is less toxic than the wild type diphtheria toxin A.

20. A vector comprising the nucleic acid molecule of claim 18.
